(19) United States
(12) Patent Application Publication
(10) Pub. No.: US 2020/0157626 A1
(43) Pub. Date: May 21, 2020

(54) METHOD FOR DIAGNOSING OR MONITORING CONDITIONS CHARACTERIZED BY ABNORMAL TEMPORAL VARIATIONS AND METHOD OF NORMALIZING EPIGENETIC DATA TO COMPENSATE FOR TEMPORAL VARIATIONS

(71) Applicants: Steven D. Hicks, Hershey, PA (US); Frank A. Middleton, Fayetteville, NY (US); Richard Uhlig, Ithaca, NY (US); QUADRANT BIOSCIENCES INC., Syracuse, NY (US); THE RESEARCH FOUNDATION FOR THE STATE UNIVERSITY OF NEW YORK, Syracuse, NY (US); PENN STATE RESEARCH FOUNDATION, University Park, PA (US)

(72) Inventors: Steven D. HICKS, Hershey, PA (US); Frank A. MIDDLETON, Fayetteville, NY (US); Richard UHLIG, Ithaca, NY (US)

(73) Assignees: Quadrant Biosciences Inc., Syracuse, NY (US); The Research Foundation for the State University of New York, Syracuse, NY (US); Penn State Research Foundation, University Park, PA (US)

(21) Appl. No.: 16/496,190

(22) PCT Filed: Mar. 20, 2018
(86) PCT No.: PCT/US2018/023336

§ 371 Date: Sep. 20, 2019

Related U.S. Application Data
(60) Provisional application No. 62/475,705, filed on Mar. 23, 2017.

(51) Int. Cl. C12Q 1/6883; G16B 25/10

(52) U.S. Cl. CPC C12Q 1/6883; G16B 25/10; G01N 2496/00; G01N 2800/52; G01N 2800/2864; C12Q 2600/178

(57) ABSTRACT

Methods for diagnosing or monitoring a condition, disorder or disease associated with circadian, diurnal or other temporal rhythms by detecting circa-miRNAs and circa-microbiomes associated with said condition, disorder or disease. Methods for correcting or normalizing sequence data to correct for diurnal or circadian fluctuations in quantities of circa-miRNAs and/or circa-microbiomes by adjusting or normalizing values based on the time of day when a saliva sample containing these RNAs was collected.

### CROSS-REFERENCE TO RELATED APPLICATIONS

This application claims priority to U.S. Provisional 62/475,705 (Oblon 503568US) entitled METHOD OF NORMALIZING EPIGENETIC DATA TO ACCOUNT FOR TEMPORAL VARIATIONS, filed Mar. 23, 2017.

### GOVERNMENT RIGHTS

This invention was made with government support under Grant No. 1 R41 MH111347-01 awarded by the National Institute of Health (NIH) under the Small Business Technology Transfer Grant program. The government has certain rights in the invention.

### BACKGROUND OF THE INVENTION

### Field of the Invention

The invention involves methods for correcting or normalizing values of salivary mi-RNA and/or microbial RNA levels to compensate for temporal variations, such as circadian fluctuations, in salivary RNA levels. Detection of abnormal temporal variations in salivary mi-RNA and/or microbial RNA levels that correlate with a disease, injury or other disorder or with health status.

### Description of Related Art

The proper regulation of sleep in humans is critical for normal mental and physical health. Most major organ systems exhibit fluctuations in their functional state related to sleep-wake cycles or circadian rhythm [1-3]. Disturbances in sleep or disruption of circadian rhythm are a common problem in many chronic brain disorders, including autism, depression, Parkinson's, and Alzheimer's and these symptoms have a negative impact on activities of daily living [3].

During sleep-wake cycles there are numerous molecular, cellular, and physiological changes that occur. Many of these changes are driven by circadian regulatory genes, such as CLOCK and BMAL [4]. These, in turn, cause a vast array of changes in the expression of physiologically significant genes, proteins, and hormones, influencing nearly every body system. However, apart from light-dark cycles, the factors that influence expression of circadian rhythm are not fully understood.

MicroRNAs (“miRNAs”) are small, noncoding RNA fragments, approximately 20-22 nucleotides long in their mature state. MiRNAs are involved in post-transcriptional regulation of gene expression [5-8]. After processing by endonucleases [8, 9], single-stranded miRNAs combine with other macromolecules to form RNA-induced silencing complexes or RISCs. RISCs target complementary messenger RNA (“mRNA”) strands for degradation and interfere with their translation, thereby altering cellular function [8, 9]. MiRNAs exert widespread influence on gene expression. More than 1,900 identified miRNAs have been shown to affect the expression of up to 60% of all genes [10-13]. MiRNAs play a role in virtually all cellular functions, such as cell proliferation, differentiation, and apoptosis [6, 10, 11].

MiRNAs are found in nearly all body cells, tissues, and biofluids [10, 14]. Because miRNAs regulate the majority of human genes, a considerable number of genes associated with the circadian cycle are now thought to be directly under their influence, including CLOCK and BMAL, among others [15]. MiRNAs that circulate throughout the body in extracellular fluids are resistant to enzymatic degradation [16], and thus may act as critical components of a molecular endocrine system [17]. Indeed, there are now considerable data implicating miRNAs in the control of various endocrine and metabolic tissues, such as the pineal and pituitary glands [18], the hypothalamus, and the gastrointestinal (GI) tract. Furthermore, disruption of circadian regulation by miRNAs can lead to significant pathology [19].

Notably, the activities of miRNAs in the gut appear to extend beyond the regulation of host gene expression and include a strong relationship with the resident bacteria of the microbiome [20, 21]. Within the GI system, the microbiome contributes to energy harvesting by generating numerous metabolites and intermediates that influence the function of other organ systems, including the brain and endocrine organs [22]. Recent evidence also indicates that there are circadian changes in the gut microbiome [23]. Thus, cross-talk between host miRNAs and the GI microbiome may work in concert to influence temporal changes in gene expression that drive host behavior and disease.

Only one prior study has demonstrated diurnal variations for a select number of cell free microRNAs in human plasma using quantitative RT-PCR [24]. However, no prior studies have harnessed next-generation sequencing to investigate diurnal variations for the entire micro-transcriptome or explored these diurnal patterns in the GI tract parallel to the microbiome.

In view of the above, the inventors investigated whether a saliva-based collection method could identify host miRNA and microbial RNA elements that manifested consistent and parallel circadian oscillations; whether these RNA elements would target functionally-relevant biologic pathways related to host immunity, circadian rhythm, and metabolism; and whether a subset of circadian miRNAs could demonstrate “altered” expression in a cohort of children with disordered sleep patterns.

### BRIEF SUMMARY OF THE INVENTION

A method for normalizing data representing miRNA and/or microbial RNA concentrations in saliva comprising (i) obtaining saliva samples containing miRNA and/or microbial RNA at the same time each day; or (ii) determining whether the concentration of a salivary miRNA and/or microbial RNA exhibits a circadian rhythm and when such a circadian rhythm is determined, normalizing the values of miRNA and/or microbial RNA levels taken at different times during the day based on the circadian rhythm determined for that RNA.

When a miRNA or microbial RNA detection method is performed by measuring the amounts of two or more miRNAs or microbial RNAs, each of the RNAs may be normalized based on its own pattern of circadian expression over a day. Normalized levels or patterns of expression of different miRNAs and/or microbial RNAs that exhibit circadian or other temporal rhythms in their concentrations in saliva may then be compared or associated with particular symptoms without variations introduced by measurement at different points in time.

A miRNA and/or microbial RNA level may be normalized (internally) to a value taken for that same RNA at a particular time of day or may be normalized (externally) to a value from a different invariant miRNA and/or invariant microbial RNA whose salivary level is constant and does not fluctuate over the day.

A method for detecting a disease, injury or other disorder associated with a disrupted or irregular or other abnormal temporal or circadian rhythm of miRNA and/or microbial RNA levels by detecting levels of at least one salivary miRNA and/or microbial RNA. This method may measure depression or elevation the level of expression of a miRNAs or microbial RNAs that ordinarily does not vary over the day (e.g., an “invariant miRNA” or “invariant microbial RNA” that is constitutively expressed and exhibits a constant concentration in saliva over the day) or measure disruptions in the normal circadian rhythm of a level of a miRNA (e.g., a CircamiR) and/or microbial RNA (e.g., a CircaMicrobe RNA).

Parallel circadian oscillation in host and microbial RNA represents an important consideration for studies analyzing epi-transcriptomic or metagenomic mechanisms in human health and disease. Circadian rhythm disturbances are a common problem in disorders of the central nervous system (e.g. Parkinson's, Alzheimer's, autism, depression, concussion [47]). Hence, studies of peripheral miRNA expression in these conditions might consider how diurnal miRNA expression patterns are shifted, rather than simply focusing on average miRNA levels at a single collection point in comparison with a control cohort. Monitoring levels of these factors in biofluids like saliva could have diagnostic potential in diseases with altered circadian rhythm and may one day provide a basis for targeted miRNA therapy of circadian disruptions.

### BRIEF DESCRIPTION OF THE SEVERAL VIEWS OF THE DRAWING(S)

The patent or application file contains at least one drawing executed in color. FIG. 1. Flow chart outlining an analytic approach. Sample Sets 1 and 2 were used to identify circadian RNA candidates (green), which were then validated in sample Set 3 (blue). Relationships between CircaMiR levels and CircaMicrobes, or mRNA targets were explored (orange). The functional implications of CircaMiRs and CircaMicrobes were interrogated through annotation analyses and characterization in a cohort of children with disordered sleep (sample Set 4; red). The relationship of oscillating RNA with patterns of daily activity (sleep, eating, and tooth brushing) were also investigated.

FIG. 2A: Heat map clustering of expression data for the 61 miRNAs changed according to collection time in sample Set 1. This set consisted of 24 samples from 4 subjects across 3 days of sampling (days 1, 3, 7) at a frequency of 2 times/day (9 am, 9 pm).

FIG. 2B: Heat map clustering of expression data for the 61 miRNAs changed according to collection time in sample Set 2. This set consisted of 48 samples from 3 subjects obtained across 4 days of sampling (days 1, 5, 10, 15) at a frequency of 4 times/day (9 am, 1:30 pm, 5:30 pm, 9 pm).

FIG. 2C (FIG. 3 from 62/475,705) shows a heat map clustering of expression data for the 19 miRNAs changed according to collection time in 24 samples from 4 subjects across 3 days of sampling (days 1, 3, 7) at a frequency of 2 times/day (8 am, 8 pm).

FIG. 2D (FIG. 3 from 62/475,705) shows a heat map clustering of expression data for the 19 miRNAs changed according to collection time in 48 samples from 3 subjects across 4 days of sampling (days 1, 5, 10, 15) at a frequency of 4 times/day (8 am, 12 pm, 4 pm, 8 pm).

FIG. 3A. 11 of the total 61 identified miRNA predictors and their accuracy of prediction for sample Set 3.

FIG. 3B. Sine transformed values of the average expression of 1 of the 61 CircaMiRs (miR-199b-3p) for the subjects in sample Set 3 (collected at various times across 2 days).

FIG. 3C (FIG. 5 from 62/475,705) shows normalized data for 1 of the top 19 miRNAs shown for 3 of the subjects in Collection 3 (collected at various times).

FIG. 4. A Pearson's correlation analysis was used to determine relationships between the 11 CircaMiRs and 11 CircaMicrobes. The 22 RNA features are sorted by a complete clustering algorithm, and the hierarchical tree indicates similarity in expression pattern across samples. Blue indicates strong inverse relationships while red indicates strong direct relationships.

FIG. 5A. Changes in functional microbiome expression across time. The hierarchical heat map displays average abundance values for microbial RNAs representing 22 KEGG/COG metabolic pathways, that displayed nominal differences (p<0.05) in expression across 4 time periods (7-9 AM, 10 AM-2 PM, 3-6 PM, 7-10 PM). The dendrogram (y-axis) represents inter-relatedness of KEGG/COG pathway activity measured by Pearson distance metric across the 120 samples. Red denotes relative increased abundance of KEGG/COG transcripts, while blue denotes relative decrease in related transcripts. Chi-square and raw p-values (Kruskal-Wallis ANOVA) are displayed for each of the 22 pathways.

FIG. 5B. Changes in functional microbiome expression across time. A partial least squares discriminant analysis utilizing mean abundance levels for all 202 KEGG/COG metabolic pathways with microbial RNA mappings is displayed for the four collection time periods. Note that global metabolic activity in these 202 pathways achieves partial separation of the four time periods, while accounting for 20.6% of the variance in the dataset.

FIG. 6. A two-way ANOVA assessed relationships between levels of 14 CircaMiRs, collection time, and the presence/absence of disordered sleep in a cohort of 140 children with autism spectrum disorder. The Venn diagram (center) shows that 7/14 (50%) of theses CircaMiRs displayed significant relationships with collection time, disordered sleep, or a time-sleep interaction. Mean expression level at 6 time points (8-9 a.m., 10-11 a.m., 12-1 p.m., 2-3 p.m., 4-5 p.m., and 6-8 p.m.) is displayed for participants with (red), or without (blue) disordered sleep for each of the 7 CircaMiRs of interest. Two-way ANOVA p-values are listed for each CircaMiR in the embedded table (center, bottom).

FIG. 7. Multivariate regression with 11 CircaMiRs demonstrates significantly utility for predicting time of collection in an independent sample of 63 children with autism spectrum disorder (ASD) who had normal sleep patterns. The graph plots the relationship between the predicted time and actual time in hours. The lines above and below the regression line indicate the 95th confidence interval of the fitted regression. The colored ellipse represents the 95th confidence interval of the actual data points. There was an absence of a significant relationship in ASD children with a sleep disorder.

FIG. 8 shows the synthesis and extracellular release of miRNA. miRNAs are transcribed from DNA in the nucleus and processed by key enzymes such as Drosha and Dicer into their mature form that influences protein translation in the RNA-Induced Silencing Complex (RISC). Cells also have the ability to release miRNA into the extracellular fluids, such as saliva, within exosomes derived from multi-vesicular bodies (MVB), microvesicles, or bound to proteins such as high density lipoprotein (HDL).

FIG. 9 shows a Venn diagram of overlapping miRNAs from analysis of 24 samples in Collection 1 and 48 samples in Collection 2.

FIG. 10 shows a heat map clustering of expression data for the 19 miRNAs changed according to collection time in 24 samples from 4 subjects across 3 days of sampling (days 1, 3, 7) at a frequency of 2 times/day (8 am, 8 pm)

FIG. 11 shows a heat map clustering of expression data for the 19 miRNAs changed according to collection time in 48 samples from 3 subjects across 4 days of sampling (days 1, 5, 10, 15) at a frequency of 4 times/day (8 am, 12 pm, 4 pm, 8 pm).

FIG. 12 shows normalized data for 1 of the top 19 miRNAs shown for 3 of the subjects in Collection 3 (collected at various times).

FIG. 13 shows absolute abundance of species in the microbiome of one of the subjects in Collection 3.

FIG. 14 shows a Venn diagram of overlapping significantly changed microbes from analysis of Collection 1 and 2 samples.

FIGS. 15A-15D show a Pearson correlation matrix of circadian microbes and circaMiRs. Note the presence of several large correlations between the circaMiRs and microbes (lower left, upper right).

FIG. 16 shows 45 genes involved in Circadian Rhythm Signaling were identified as targets of 14 of the circaMiRs. This is almost one-third of the 139 total annotated genes involved in circadian function in IPA. In the figure, genes targeted by 1 miRNA are highlighted and gray, while genes targeted by >1 of the 14 miRNAs are highlighted and red. Untargeted genes appear as white.

### DETAILED DESCRIPTION OF THE INVENTION

The microbiome plays a vital role in human health and disease. Interaction between human hosts and the microbiome occurs through a number of mechanisms, including transcriptomic regulation by microRNA (miRNA). In animal models, circadian variations in miRNA and microbiome elements have been described, but patterns of co-expression and potential diurnal interaction in humans have not. We investigated daily oscillations in salivary miRNA and microbial RNA to explore relationships between these components of the gut-brain-axis and their implications in human health. Nine subjects provided 120 saliva samples at designated times, on repeated days. Samples were divided into three sets for exploration and cross-validation. Identification and quantification of host miRNA and microbial RNA was performed using next generation sequencing. Three stages of statistical analyses were used to identify circadian oscillators: 1) a two-way analysis of variance in the first two sample sets identified host miRNAs and microbial RNAs whose abundance varied with collection time (but not day); 2) multivariate modeling identified subsets of these miRNAs and microbial RNAs strongly-associated with collection time, and evaluated their predictive ability in an independent hold-out sample set; 3) regulation of circadian miRNAs and microbial RNAs was explored in data from autistic children with disordered sleep (n=77), relative to autistic peers with typical sleep (n=63). Eleven miRNAs and 11 microbial RNAs demonstrated consistent diurnal oscillation across sample sets and accurately predicted collection time in the hold-out set. Associations among five circadian miRNAs and four circadian microbial RNAs were observed. We termed the 11 miRNAs CircaMiRs. These CircaMiRs had 1,127 predicted gene targets, with enrichment for both circadian gene targets and metabolic signaling processes. Four CircaMiRs had “altered” expression patterns among children with disordered sleep. Thus, novel and correlated circadian oscillations in human miRNA and microbial RNA exist and may have distinct implications in human health and disease.

Saliva is a slightly alkaline secretion of water, mucin, protein, salts, and often a starch-splitting enzyme (as ptyalin) that is secreted into the mouth by salivary glands, lubricates ingested food, and often begins the breakdown of starches. Saliva is released by the submandibular gland, parotid gland, and/or sublingual glands and saliva release may be stimulated by the sympathetic and/or parasympathetic nervous system activity. Saliva released primarily by sympathetic or parasympathetic induction may be used to isolate microRNAs.

Saliva may be collected by expectoration, swabbing the mouth, passive drool, or by other methods known in the art. It can be collected from the mouth prior to or after a rinse, brushing, mouthwash or food intake. For example, in some embodiments it may be collected without rinsing the mouth first and in other embodiments after rinsing accumulated saliva out of the mouth and collecting newly secreted saliva, optionally after the administration of a sialagogue, such as a parasympathomimetic drug (e.g., pilocarpine) acting on parasympathetic muscarinic receptors, such as the M3 receptor, to induce an increased saliva flow. Malic acid, ascorbic acid, chewing gum or plant or herbal extracts that promote saliva flow may also be used. In other embodiments saliva may be withdrawn from a salivary gland.

In some embodiments, a saliva sample may be further purified by centrifugation, filtration, or other means that preserves miRNA content. For example, it may be filtered through a 0.22 micron or 0.45 micron membrane and the separated components, such as cells, microvesicles, or fluids used to recover microRNAs or microbial RNAs.

In other embodiments, proteins or enzymes that degrade microRNA may be removed, inactivated or neutralized in a saliva sample, for example, a RNAse inhibitor such as Superase In RNase Inhibitor, may be added to a sample containing miRNA.

MicroRNA or miRNA is a small non-coding RNA molecule containing about 22 nucleotides, which is found in plants, animals and some viruses, that functions in RNA silencing and post-transcriptional regulation of gene expression; see Ambros, V (Sep. 16, 2004). The functions of animal microRNAs. Nature. 431 (7006): 350-5. doi:10.1038/nature02871. PMID 15372042; or Bartel, D P (Jan. 23,2004). MicroRNAs: genomics, biogenesis, mechanism, and function. Cell. 116 (2): 281-97. doi: 10.1016/S0092-8674(04)00045-5. PMID 14744438, both of which are incorporated by reference.

A miRNA standard nomenclature system uses the prefix “miR” followed by a dash and a number, the latter often indicating order of naming. For example, miR-120 was named and likely discovered prior to miR-241. A capitalized “miR-” refers to the mature form of the miRNA, while the not capitalized “mir-” refers to the pre-miRNA and the pri-miRNA, and “MIR” refers to the gene that encodes them. The prefix “hsa-” denotes a miRNA from humans.

Microbial RNA is RNA produced by microbes such as those present in the oral cavity. It may be collected from saliva by procedures similar to those described above for miRNA.

miRNA or microbial RNA isolation from biological samples such as saliva and their analysis may be performed by methods known in the art, including the methods described by Yoshizawa, et al., Salivary MicroRNAs and Oral Cancer Detection, Methods Mol Biol. 2013; 936: 313-324; doi: 10.1007/978-1-62703-083-0 (incorporated by reference) or by using commercially available kits, such as mirVana™ miRNA Isolation Kit which is incorporated by reference to the literature available at https://_tools.thermofisher.com/content/sfs/manuals/fm_1560.pdf (last accessed Jan. 30, 2018).

Mimics. In some embodiments, miRNA mimics may be employed. Such mimics may be small, double-stranded RNA molecules designed to mimic endogenous mature miRNA molecules once transfected into a cell. Mimics may target and modulate the expression of the same gene(s) as the corresponding native miRNA or may be designed to have lower, higher, or altered activity on target gene(s). Mimics are often used for gene silencing. They generally contain a sequence at least partially complementary to a three prime untranslated region (3′-UTR) of a target gene or sequence. A seed sequence that targets a miRNA to a particular RNA generally contains 6-8 nucleotides complementary to a target RNA sequence. A mimic may comprise the same seed sequence as an miRNA described herein.

Some miRNA mimics may contain non-natural nucleotides. Artificial nucleic acids such as locked nucleic acids (“LNAs”) or bridged nucleic acids (“BNAs”) may be used as mimics. Such mimics are commercially available; see http://_www.biosyn.com/bna-synthesis-bridged-nucleic-acid.aspx (last accessed Jan. 22, 2018, incorporated by reference).

Such miRNA mimics may be designed based on information available in the miRBase; http://_www.mirbase.org/ (ver. 21) (last accessed Jan. 22, 2018) which is incorporated by reference.

Next Generation Sequencing refers to non-Sanger-based high-throughput DNA sequencing technologies. Millions or billions of DNA strands can be sequenced in parallel, yielding substantially more throughput and minimizing the need for the fragment-cloning methods that are often used in Sanger sequencing of genomes. Next generation sequencing methods useful for sequencing miRNA and microbial RNAs are known and incorporated by reference to https://_en.wikipedia.org/wiki/DNA_sequencing (last accessed Jan. 30, 2018).

DIANA-mirPath is a miRNA pathway analysis web-server, providing accurate statistics, while being able to accommodate advanced pipelines. mirPath can utilize predicted miRNA targets (in CDS or 3′-UTR regions) provided by the DIANA-microT-CDS algorithm or even experimentally validated miRNA interactions derived from DIANA-TarBase. These interactions (predicted and/or validated) can be subsequently combined with sophisticated merging and meta-analysis algorithms; see Vlachos, Ioannis S., Konstantinos Zagganas, Maria D. Paraskevopoulou, Georgios Georgakilas, Dimitra Karagkouni, Thanasis Vergoulis, Theodore Dalamagas, and Artemis G. Hatzigeorgiou. DIANA-miRPath v3.0: deciphering microRNA function with experimental support. Nucleic acids research (2015): gkv403 (incorporated by reference) and http://_snf-515788.vm.okeanos.grnet.gr/ (last accessed Jan. 25, 2018, incorporated by reference.

MicrobiomeAnalyst is software that provides comprehensive statistical, visual and meta-analysis of microbiome data; see http://_www.microbiomeanalyst.ca/faces/home.xhtml (incorporated by reference; last accessed Jan. 31, 2018).

MetaboAnalyst is a comprehensive tool for metabolomics analysis and interpretation; see http://_www.metaboanalyst.ca/ (incorporated by reference; last accessed Jan. 31, 2018).

Ingenuity® Pathway Analysis is an analysis and search tool that uncovers the significance of ‘omics data and identifies new targets or candidate biomarkers within the context of biological systems; see https://_www.qiagenbioinformatics.com/products/ingenuity-pathway-analysis/ (incorporated by reference, last accessed Jan. 31, 2018).

Normalization. Sequence Read counts also can be normalized based on known methods. For example, normalization methods for RNA sequence data may be used as described by Li, et al., BMC Informatics 16:347, Comparing the normalization methods for the differential analysis of Illumina high-throughput RNA-Seq data (2015; incorporated by reference). Normalization can be used to provide more accurate identification of relative concentrations of different miRNAs or microbeRNAs. Normalization based on a level or average levels of one or more invariant miRNAs or RNAs (RNAs that do not substantially fluctuate in level or concentration over a 24 hour day or other repeated temporal period) may also be used to normalize sequence read counts and to calculate absolute quantification of miRNA or microbe RNA. Normalization may be based on a global mean miRNA expression normalizer (majority of miRNAs which remain invariable). Normalization procedures for miRNA sequence reads, such as those obtained from NextGen Sequencing are also described and incorporated by reference to https://_en.wikipedia.org/wiki/MicroRNA_sequencing.

Normalization to compensate for RNA concentration variations tied to a diurnal or circadian cycle. Amounts of a miRNA or microbial RNA for particular miRNA or microBIOME RNA may vary at different times of day, for example, for CircaMiRNAs and CircaMicrobe RNAs. To avoid introducing error due to fluctuation over a time period saliva samples may be taken at the same time each day or at the same time with respect to the relevant repeating time period. This is often not practical in a clinical setting. Moreover, measuring miRNA or salivary microBiome RNA levels at an arbitrary time only provides information about miRNA level or relative miRNA levels (or microBiome RNA) levels at that time of day, when measurements at other or additional times during the day may provide data better associated with particular conditions, disorders or diseases. By analogy, non-fasting measurement of blood sugar level generally provides a substantially different blood sugar level value than measurement of a fasting level. Similarly, measurement of miRNA or microBiome RNA at one time of the day may not reflect important correlations with a particular condition, disorder or disease.

By characterizing the cyclic patterns of CircaMiRNAs and CircaMicrobe RNAs the inventors provide a convenient way to minimize or avoid errors due to cyclic fluctuations in such RNAs. Quantities of CircaMiRNAs or CircaMicrobe RNAs may be normalized to those at a particular time in the cycle.

Based on the identification of cyclic patterns of circa-miRNAs and circa-Microbe RNAs obtained by the inventors the values of salivary miRNA and microbial RNA concentrations collected at different times of day can be more reliably and accurately compared. Using these data describing cyclic expression patterns for a variety of different miRNAs, these levels may be normalized for easy comparison and for association with particular conditions, disorders or conditions, such as those associated with mRNAs targeted by particular miRNAs. For example, amounts of a particular kind of miRNA measured at noon, 6 p.m. and midnight may be expressed as percentages of the amount of miRNA X measured at a reference time of 6 a.m. Normalization may be based on comparing a concentration of a miRNA and/or microbial RNA collected at a particular time of day with a level of a RNA expressed by one or more housekeeping genes (or an average of several housekeeping genes).

Other factors may also be used to normalize and help compare miRNA and/or microbial RNA levels throughout the day, including comparison to RNA amounts immediately before or after sleep (or at a set pre- or post-sleep interval), immediately before or after a meal (or at a set pre- or post-prandial interval), or immediately before and after exercise (e.g., at a resting heart rate time, or a non-resting heart rate time) or at a set pre- or post-exercise interval. Pre- and post-intervals above may range from 1, 2, 5, 10, 15, 30, 60, 90, 120, 240 or >240 mins or any intermediate value within this range.

Other factors may also be normalized to improve data quality or facilitate analysis or comparison. A subject's epigenetic and/or microbiome genetic sequence data may be normalized to account for inter-sample count variations; such count normalization utilizing one or more invariant miRNAs and/ or microbial RNAs so as to represent data in proportion to their relative expression. Normalization methods for RNA sequence data may also be used; see the methods described by Li, et al., BMC Informatics 16:347, Comparing the normalization methods for the differential analysis of Illumina high-throughput RNA-Seq data (2015; incorporated by reference).

In some embodiments raw miRNA or other RNA read counts within each sample are separately quantile normalized, mean-centered, and divided by the standard deviation of each variable prior to statistical analysis and whisker box plots of quantile normalized abundance are prepared.

In other embodiments, miRNA data and microbiome may be separately normalized to control for differences in total read number and subjected to quantile normalization. Normalized values may then be screened for sphericity prior to statistical analysis using principle component analysis (“PCA”). Data can be then filtered to eliminate those with more than 60% missingness and to remove extreme outlier samples based on the PCA results. Errors due to differences in the time-of-day collection for circa-miRNAs and CircaMicrobeMicrobe RNA expression data may be removed by normalizing scaling or otherwise accounting for the values of the data based on observed temporal fluctuations in levels of these RNAs. A non-parametric Mann-Whitney test may be initially used to screen for the most robust miRNA and microbiome taxon IDs having significant impact on diagnosis of a condition, disorder or disease. The top significant miRNAs and taxon IDs may then be used to product a diagnostic classification model and generate a correlation matrix. MiRNAs that show the strongest predictive utility can then be subjected to functional analysis using Diana Tools miRpath.

miR-RNA and RNA assays. The mi-RNAs described herein may be detected using conventional miRNA and RNA assays. Conventional methods for detecting miRNA include Northern blot analysis; detection-based hybridization using microarrays; a method of detecting and quantifying a certain miRNA by a two-step process comprising RT-PCR, which uses stem-loop primers binding complementarily to the miRNA, and subsequent quantitative PCR (Chen et al., Nucleic Acids Res., 33(20): e179, 2005); and a method comprising tailing the 3′-end of miRNA with poly(A) using a poly(A) polymerase, synthesizing cDNA using a poly(T) adaptor as a primer, and then amplifying the miRNA using a miRNA-specific forward primer and a reverse primer based on the poly(T) adaptor (Shi, R. and Chiang, V. L., BioTechniques, 39: 519-525, 2005). High-throughput microarrays have been developed to identify expression patterns for miRNAs in a variety of tissue and cell types (see, e.g., Babak et al., RNA 10:1813 (2004); Calin et al., Proc. Natl. Acad. Sci. USA 101:11755 (2004); Liu et al., Proc. Natl. Acad. Sci. USA 101:9740 (2004); Miska et al., Genome Biol. 5:R68 (2004); Sioud and ROsok, BioTechniques 37:574 (2004); Krichevsky et al., RNA 9:1274 (2003)). A circadian rhythm is any biological process that displays an endogenous, entrainable oscillation of about 24 hours. These 24-hour rhythms are driven by a circadian clock, and they have been widely observed in plants, animals, fungi, and cyanobacteria. The study of biological temporal rhythms, such as daily, tidal, weekly, seasonal, and annual rhythms, is called chronobiology. Processes with 24-hour oscillations are usually called diurnal rhythms; strictly speaking, they should not be called circadian rhythms unless their endogenous nature is confirmed. Although circadian rhythms are endogenous (“built-in”, self-sustained), they are adjusted (entrained) to the local environment by external cues called, which include light, temperature and redox cycles. In medical science, an abnormal circadian rhythm in humans is known as circadian rhythm disorder.

The glymphatic system or glymphatic clearance pathway is a functional waste clearance pathway for the vertebrate central nervous system (CNS) active at night in healthy individuals. It may be subject to regulation by miRNA levels or it may contribute to levels of miRNA associated with diurnal, circadian, or other temporal rhythms. The pathway conrIA a para-arterial influx route for cerebrospinal fluid (CSF) to enter the brain parenchyma, coupled to a clearance mechanism for the removal of interstitial fluid (ISF) and extracellular solutes from the interstitial compartments of the brain and spinal cord. Exchange of solutes between the CSF and the ISF is driven by arterial pulsation and regulated during sleep by the expansion and contraction of brain extracellular space. Clearance of soluble proteins, waste products, and excess extracellular fluid is accomplished through convective bulk flow of the ISF, facilitated by astrocytic aquaporin 4 (AQP4) water channels; See Jessen N A, Munk A S, Lundgaard I, Nedergaard M. The Glymphatic System: A Beginner's Guide. Neurochem Res. 2015;40(12):2583-99 which is incorporated by reference. Proper function of the glymphatic system has been found necessary to removal of soluble amyloid beta and thus its dysfunction may play a role in neurodegenerative proteinopathies such as amyotrophic lateral sclerosis, Alzheimer's disease, Parkinson's disease and Huntington's disease. The glymphatic system may also be impaired after a brain injury such as ischemic stroke, intracranial hemorrhage or subarachnoid hemorrhage.

A sleep disorder or somnipathy is a medical disorder of the sleep patterns of a person or animal. Some sleep disorders are serious enough to interfere with normal physical, mental, social and emotional functioning. Polysomnography and actigraphy are tests commonly ordered for some sleep disorders. Common sleep disorders include The most common sleep disorders include: Bruxism, involuntarily grinding or clenching of the teeth while sleeping. Catathrenia, nocturnal groaning during prolonged exhalation. Delayed sleep phase disorder (DSPD), inability to awaken and fall asleep at socially acceptable times but no problem with sleep maintenance, a disorder of circadian rhythms. Other such disorders are advanced sleep phase disorder (ASPD), non-24-hour sleep-wake disorder (non-24) in the sighted or in the blind, and irregular sleep wake rhythm, all much less common than DSPD, as well as the situational shift work sleep disorder. Hypopnea syndrome, abnormally shallow breathing or slow respiratory rate while sleeping. Idiopathic hypersomnia, a primary, neurologic cause of long-sleeping, sharing many similarities with narcolepsy. Insomnia disorder (primary insomnia), chronic difficulty in falling asleep and/or maintaining sleep when no other cause is found for these symptoms. Insomnia can also be comorbid with or secondary to other disorders. Kleine-Levin syndrome, a rare disorder characterized by persistent episodic hypersomnia and cognitive or mood changes. Narcolepsy, including excessive daytime sleepiness (EDS), often culminating in falling asleep spontaneously but unwillingly at inappropriate times. About 70% of those who have narcolepsy also have cataplexy, a sudden weakness in the motor muscles that can result in collapse to the floor while retaining full conscious awareness. Night terror, Pavor nocturnes, sleep terror disorder, an abrupt awakening from sleep with behavior consistent with terror. Nocturia, a frequent need to get up and urinate at night. It differs from enuresis, or bed-wetting, in which the person does not arouse from sleep, but the bladder nevertheless empties. Parasomnias, disruptive sleep-related events involving inappropriate actions during sleep, for example sleep walking, night-terrors and catathrenia. Periodic limb movement disorder (PLMD), sudden involuntary movement of arms and/or legs during sleep, for example kicking the legs. Also known as nocturnal myoclonus. See also Hypnic jerk, which is not a disorder. Rapid eye movement sleep behavior disorder (RBD), acting out violent or dramatic dreams while in REM sleep, sometimes injuring bed partner or self (REM sleep disorder or RSD). Restless legs syndrome (RLS), an irresistible urge to move legs. RLS sufferers often also have PLMD. Shift work sleep disorder (SWSD), a situational circadian rhythm sleep disorder. Jet lag was previously included as a situational circadian rhythm sleep disorder, but it doesn't appear in DSM-5 (see Diagnostic and Statistical Manual of Mental Disorders). Sleep apnea, obstructive sleep apnea, obstruction of the airway during sleep, causing lack of sufficient deep sleep, often accompanied by snoring. Other forms of sleep apnea are less common.^{[X]} When air is blocked from entering into the lungs, the individual unconsciously gasps for air and sleep is disturbed. Stops of breathing of at least ten seconds, 30 times within seven hours of sleep, classifies as apnea. Other forms of sleep apnea include central sleep apnea and sleep-related hypoventilation. Sleep paralysis, characterized by temporary paralysis of the body shortly before or after sleep. Sleep paralysis may be accompanied by visual, auditory or tactile hallucinations. Not a disorder unless severe. Often seen as part of narcolepsy. Sleepwalking or somnambulism, engaging in activities normally associated with wakefulness (such as eating or dressing), which may include walking, without the conscious knowledge of the subject. Somniphobia, one cause of sleep deprivation, a dread/ fear of falling asleep or going to bed. Signs of the illness include anxiety and panic attacks before and during attempts to sleep. Other sleep disorders are incorporated by reference to https://_en.wikipedia.org/wiki/Sleep_disorder (last accessed Jan. 29, 2018).

Some nonlimiting embodiments of the invention include the following:

1. A method for normalizing epigenetic and/or microbiome genetic data to account for temporal variations in microRNA (“miRNA”) expression levels and/or microbiome RNA expression levels, the method comprising:

(a) determining read-counts for one or more miRNAs and/or read-counts from microbiome RNA sequences in a biological sample taken from a subject,

(b) normalizing said read-counts to account for inter-sample read-count variations by utilizing read-counts from one or more invariant or constitutively expressed miRNAs or genes to provide inter-sample read-count normalized data,

(c) determining a time of day that the biological sample was taken, and

(d) further normalizing the inter-sample read-count normalized data based on the time of day the biological sample was taken by applying an algorithm that compensates for temporal variations in the concentrations of one or more miRNAs and/or microbiome RNA expression levels, thereby providing data describing inter-sample and time-of-day normalized concentrations or levels of miRNAs and/or microbiome RNA expression levels. Preferably the biological sample is saliva, however, other biological samples such as plasma, serum, CSF, tears, nasal fluids and other mucosal secretions, prostatic fluid, sperm, urine, feces and other biological fluids or tissue samples may be used. Preferably microbiome genetic data measures overall RNA expression at particular times of day. However, the expression of RNA from one or more microbes may be used or the concentration of particular genetic markers for various microbes, such as rRNA content may be measured. Fluctuations in levels of different microorganisms (as distinguishable from fluctuations in the expression of one or more RNA expression levels) in the microbiome may also be determined by other methods known in the art, such as by determining the amount of rRNA or particular genomic markers in a saliva sample.

The method of embodiment 1 may be practiced in conjunction with one or more limitations described by embodiments 2-22.

2. A method for detecting or diagnosing a condition, disorder or disease associated with an abnormal diurnal or circadian rhythm in a human subject, the method comprising:

(a) determining a concentration level(s) of one or more micro RNAs (“miRNAs”) in a saliva sample taken from a human subject, and

(b) comparing the determined concentration level(s) of the one or more miRNAs against normal level(s) of the same one or more miRNAs in control human subject(s) not suffering from the condition, disorder of disease associated with abnormal diurnal or circadian rhythm,

(c) selecting a subject having an abnormal level of said one or more miRNAs as having or as being at higher risk for having a condition, disorder or disease associated with an abnormal diurnal or circadian rhythm;

wherein the one or more miRNAs is selected from the group consisting of miR-24-3p, miR-200b-3p, miR-203a-3p, miR-26a-5p, hsa-miR-106b-3p, hsa-miR-128-3p, hsa-miR-130a-3p, hsa-miR-15a-5p, hsa-miR-192-5p, hsa-miR-199a-3p, hsa-miR-199b-3p, hsa-miR-221-3p, hsa-miR-26b-5p, hsa-miR-3074-5p, hsa-miR-30e-3p, hsa-miR-320a, hsa-miR-345-5p, hsa-miR-375, hsa-miR-423-3p, hsa-miR-92a-3p, hsa-miR-93-5p, hsa-let-7a-5p, hsa-let-7d-3p, hsa-miR-101-3p, hsa-miR-10b-5p, hsa-miR-125b-2-3p, hsa-miR-1307-5p, hsa-miR-140-3p, hsa-miR-142-3p, hsa-miR-143-3p, hsa-miR-148b-3p, hsamiR-16-5p, hsa-miR-181a-5p, hsa-miR-181c-5p, hsa-miR-186-5p, hsa-miR-191-5p, hsa-miR-193a-5p, hsa-miR-205-5p, hsa-miR-215-5p, hsa-miR-21-5p, hsa-miR-223-3p, has-miR-22-3p, hsa-miR-23a-3p, hsa-miR-23b-3p, hsa-miR-25-3p, hsa-miR-29a-3p, hsa-miR-30d-5p, hsa-miR-320b, hsa-miR-361-5p, hsa-miR-363-3p, hsa-miR-374a-3p, hsa-miR-423-5p, hsa-miR-425-5p, hsa-miR-532-5p, hsa-miR-574-3p, hsa-miR-629-5p, hsa-miR-98-5p and/or those miRNA which share the seed sequences as the above listed miRNAs; and

wherein an abnormal level of said one or more miRNAs is indicative of the condition, disorder or disease associated with an abnormal diurnal or circadian rhythm.

3. The method of embodiment 4, wherein values of said miRNA concentration level(s) are normalized to an expression level, or average expression level, of one or more housekeeping genes whose RNA expression level is substantially invariant; and/or wherein said miRNA concentration levels are normalized to compensate for diurnal or circadian fluctuations in the expression of the one or more miRNA levels, normalized to compensate for fluctuations in the expression of the one or more miRNA levels due to food intake or exercise that raises the heart rate; or adjusted to compensate for differences in age, sex or genetic background. Housekeeping genes include those useful for calibration of RNA sequencing data such as those described by Eisenberg, et al., Trends in Genetics 29(10: 569-574, Cell Press (2013; incorporated by reference)

4. The method of embodiment 2 or 3, wherein (a) determining a concentration of one or more miRNAs is done by RNA sequencing (“RNA-seq”), qPCR, a miRNA array, or multiplex miRNA profiling. Such methods are known in the art and are also described at http://_www.abcam.com/kits/review-of-mirna-assay-methods-qper-arrays-and-sequencing (last accessed Mar. 19, 2018, incorporated by reference).

5. The method for detecting or diagnosing of embodiment 2, 3, or 4, wherein said one or more miRNAs are selected from the group consisting of miR-142-5p, miR-130b-3p, miR-629-5p, miR-140-3p, miR-128-3p, miR-181c-5p, miR345-5p, miR-22-5p, miR-8089, miR-221-3p, and miR-200b-5p.

6. The method of embodiment 2, 3, or 4, wherein the saliva sample is taken from a human subject suspected of having a sleep disorder or disordered sleep and wherein the miRNAs are selected from the group consisting of at least one of miR-24-3p, miR-200b-3p, miR-203a-3p, and miR-26a-5p.

7. The method of embodiment 2, 3, 4, 5, or 6, wherein the saliva sample is taken from the human subject at a particular time of day and the concentration level(s) of miRNA in said sample are compared to normal miRNA values in saliva taken at the same time of day under otherwise identical conditions.

8. The method of embodiment 2, 3, 4, 5, or 6, wherein the saliva sample is taken from the human subject at a different time of day than the time of day at which the normal level(s) of miRNAs were determined, further comprising adjusting or normalizing the value of the miRNA level(s) determined in the saliva sample to compensate for diurnal or circadian fluctuations in miRNA level(s).

9. The method of embodiment 2, 3, 4, 5, or 6, wherein the saliva sample is taken from the human subject at a different time of day than the time of day at which the normal level(s) of miRNAs were determined, further comprising adjusting or normalizing the value of the miRNA level(s) determined in the saliva sample to compensate for diurnal or circadian fluctuations in miRNA level(s) as determined by a regression model or other statistical analysis; or to compensate for age, sex, or genetic background.

10. The method of any one of embodiments 2-9, wherein the saliva sample is taken within 1 hour of waking, before brushing or rinsing the mouth, before eating or drinking, and/or before exercise that elevates heart rate.

11. The method of any one of embodiments 2-10, wherein said selecting comprises selecting a subject having abnormal levels of four or more of said miRNAs, and, optionally calculating a Pearson correlation coefficient of said abnormal miRNA levels with likelihood of an at least one symptom of a condition, disorder, or disease associated with an abnormal diurnal or circadian rhythm.

12. The method of any one of embodiments 2-9, wherein said selecting comprises selecting a subject having abnormal levels of ten or more of said miRNAs, and, optionally calculating a Pearson correlation coefficient of said abnormal miRNA levels with likelihood of an at least one symptom of a condition, disorder, or disease associated with an abnormal diurnal or circadian rhythm.

13. The method of any one of embodiments 2-12, further comprising determining an expression level of RNA(s) in said subject from one or more salivary microbes selected from the group consisting of Falconid herpesvirus, Prevotella melaninogenica ATCC 25845, Haemophilus parainfluenzae T3T1 Veillonella parvula DSM 2008, Macrococcus caseolyticus JSCC5402, Fusobaterium nucleatum subsp. nucleatum 25586, Haemophilus, Fusobacterium nucleatum subsp. vincentii, Mason-Pfizer monkey virus, Camplyobacer hominis ATCC, and Prevotella; or a microbe having a genome that is at least 90, 95, 96, 97, 98, 99, 99.5 or 100% similar or identical thereto; and comparing the expression level(s) of the microbial RNAs against normal level(s) of the same one or more microbial RNAs, wherein the normal (or control) expression level is that found in a subject, an average from two of more subjects, not having a condition, disorder, or disease associated with an abnormal diurnal or circadian rhythm; or concentration level(s) determined in the subject prior to appearance of one or more symptoms of a condition, disorder, or disease associated with an abnormal diurnal or circadian rhythm; and further selecting a subject having an abnormal expression level of said one or more microbial RNAs as having or as being at higher risk for having said condition, disorder or disease.

BLASTN may be used to identify a polynucleotide sequence having at least 70%, 75%, 80%, 85%, 87.5%, 90%, 92.5%, 95%, 97.5%, 98%, 99% sequence identity to a reference polynucleotide. A representative BLASTN setting optimized to find highly similar sequences uses an Expect Threshold of 10 and a Wordsize of 28, max matches in query range of 0, match/mismatch scores of 1/-2, and linear gap cost. Low complexity regions may be filtered/masked. Default settings are described by and incorporated by reference to http://blast.ncbi.nlm.nih.gov/Blast.cgi?PROGRAM=blastn&BLAST_PROGRAMS=megaBla st&PAGE_TYPE=BlastSearch&SHOW_DEFAULTS=on&LINK_LOC=blasthome (last accessed Mar. 19, 2018)(incorporated by reference).

14. The method of embodiment 2 or 13, wherein determining salivary miRNA levels or determining microbial RNA expression level(s) is done by RNA Sequencing (“RNA-Seq”).

15. The method of embodiment 14, wherein the sequencing data raw read counts are quantile-normalized, mean-centered, and divided by the standard deviation of each variable; data are normalized to account for inter-sample count variations; and/or wherein data are normalized to expression of one or more invariant miRNAs to describe relative and/or absolute expression levels; and optionally further statistically analyzing the normalized data.

16. The method of embodiment 2, further comprising treating a subject having at least one abnormal level of miRNA or microbial RNA expression level characteristic of a condition, disorder, or disease associated with an abnormal diurnal or circadian rhythm with a regimen that reduces the at least one abnormal salivary level of one or more miRNAs and/or reduces one or more abnormal microbial RNA expression levels.

17. The method of embodiment 16, further comprising obtaining saliva samples on at least two different points in time and determining efficacy of a treatment regimen when said second or subsequent saliva sample has miRNA level(s) and/or microbial RNA expression levels closer to normal.

18. The method of embodiment 2, further comprising treating a subject with a regimen that reduces at least one abnormal salivary level of one or more miRNAs or one or more abnormal microbial RNA expression levels characteristic of a condition, disorder or disease associated with an abnormal diurnal or circadian rhythm in a human subject, wherein said regimen comprises administering one or more of a sleep disorder therapy, a drug therapy, a miRNA or miRNA antagonist therapy, antimicrobial therapy, diet or nutritional therapy, phototherapy, psychotherapy, a behavior therapy, a communication therapy or an alternative medical therapy, wherein the subject was identified as having symptoms of a condition, disorder or disease associated with an abnormal diurnal or circadian rhythm.

19. An miRNA assay kit for detecting miRNAs comprising one, two or more probes or primers complementary to or otherwise suitable for amplification and/or detection of miRNAs selected from the group consisting of miR-24-3p, miR-200b-3p, miR-203a-3p, miR-26a-5p, hsa-miR-106b-3p, hsa-miR-128-3p, hsa-miR-130a-3p, hsa-miR-15a-5p, hsa-miR-192-5p, hsa-miR-199a-3p, hsa-miR-199b-3p, hsa-miR-221-3p, hsa-miR-26b-5p, hsa-miR-3074-5p, hsa-miR-30e-3p, hsa-miR-320a, hsa-miR-345-5p, hsa-miR-375, hsa-miR-423-3p, hsa-miR-92a-3p, hsa-miR-93-5p, hsa-let-7a-5p, hsa-let-7d-3p, hsa-miR-101-3p, hsa-miR-10b-5p, hsa-miR-125b-2-3p, hsa-miR-1307-5p, hsa-miR-140-3p, hsa-miR-142-3p, hsa-miR-143-3p, hsa-miR-148b-3p, hsamiR-16-5p, hsa-miR-181a-5p, hsa-miR-181c-5p, hsa-miR-186-5p, hsa-miR-191-5p, hsa-miR-193a-5p, hsa-miR-205-5p, hsa-miR-215-5p, hsa-miR-21-5p, hsa-miR-223-3p, has-miR-22-3p, hsa-miR-23a-3p, hsa-miR-23b-3p, hsa-miR-25-3p, hsa-miR-29a-3p, hsa-miR-30d-5p, hsa-miR-320b, hsa-miR-361-5p, hsa-miR-363-3p, hsa-miR-374a-3p, hsa-miR-423-5p, hsa-miR-425-5p, hsa-miR-532-5p, hsa-miR-574-3p, hsa-miR-629-5p, and hsa-miR-98-5p; reagents for amplification and/or detection of said miRNAs, and optionally a reaction substrate or platform, packaging materials and/or instructions for use.

20. The assay kit of embodiment 19 for diagnosis or detection of a sleep disorder, wherein said assay kit detects at least one of miR-24-3p, miR-200b-3p, miR-203a-3p, or miR-26a-5p.

21. The assay kit of embodiment 19 for diagnosis or detection of a sleep disorder, wherein said assay kit detects levels of miR-24-3p, miR-200b-3p, miR-203a-3p, and miR-26a-5p.

22. A method for identifying a miRNA, a concentration of which in human saliva, fluctuates according to a diurnal or circadian rhythm, comprising:
(a) collecting saliva samples from one or more subjects at 2, 3, 4, 5, 6, 7, 8, 9, 10, 11, 12 or more times or intervals during a 24 hour period,
(b) sequencing miRNA in said samples,
(c) identifying differently expressed miRNAs by counting sequencing reads per miRNA, normalizing sequence read data, and comparing normalized sequence read counts among saliva samples taken at different times,
(d) normalizing sequence read data to RNA expression of a housekeeping gene or miRNA (which exhibits invariant expression over a 24 hour period), or to an averaged RNA expression from 2, 3, 4, 5, 6, 7, 8, 9, 10 or more housekeeping genes,
(e) performing a multivariate regression analysis or other statistical analysis on the normalized RNA expression data from different time points or intervals,
(f) optionally, calculating a Pearson correlation coefficient for data obtained describing concentration levels of one or more miRNAs and one or more RNA expression levels from a microorganism found in saliva,
(g) selecting one or more miRNAs as having an expression level that fluctuates according to a diurnal or circadian rhythm; and optionally, determining target genes for miRNAs using DIANA miRpath or other software.

In another embodiment of the method of the invention described here and in the following paragraphs, is to a method for detecting an alteration in a temporal rhythm comprising: detecting at least one abnormal or altered pattern of miRNA or microbial RNA levels in saliva compared to a control value from one or more normal subjects, and selecting a subject having at least one abnormal or altered pattern of amounts of miRNA or microbial RNA; and, optionally, selecting a subject having a disease, disorder, or condition associated with an altered temporary rhythm, and optionally, administering a treatment that reduces or resynchronizes the at least one abnormal or altered pattern of amounts of the miRNA or microbial RNA. In some embodiments the temporal rhythm is a circadian or diurnal rhythm, though this method may be used to detect alterations in other kinds of temporal rhythms. The method may be used to detect alterations or abnormalities in the concentrations of 1, 2, 3, 4, 5, 6, 7, 8, 9, 10, 15, 10, 25, 50, 75, 100 or more different miRNAs.

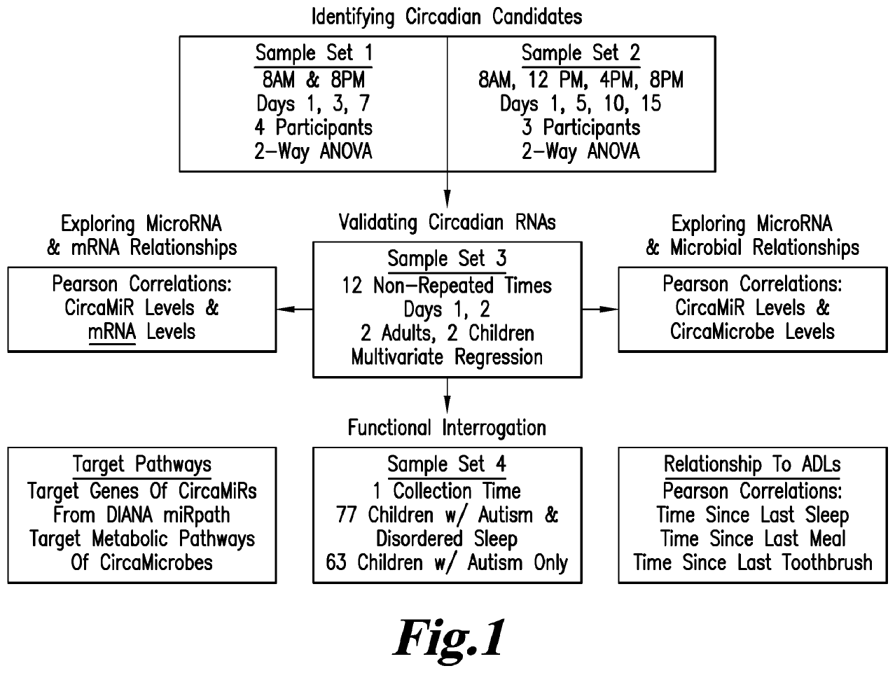

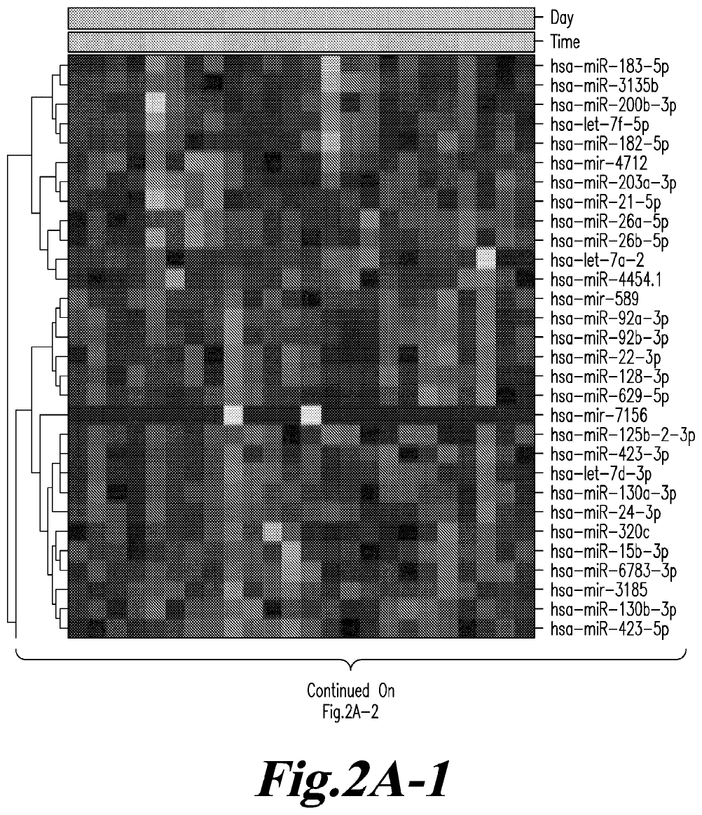

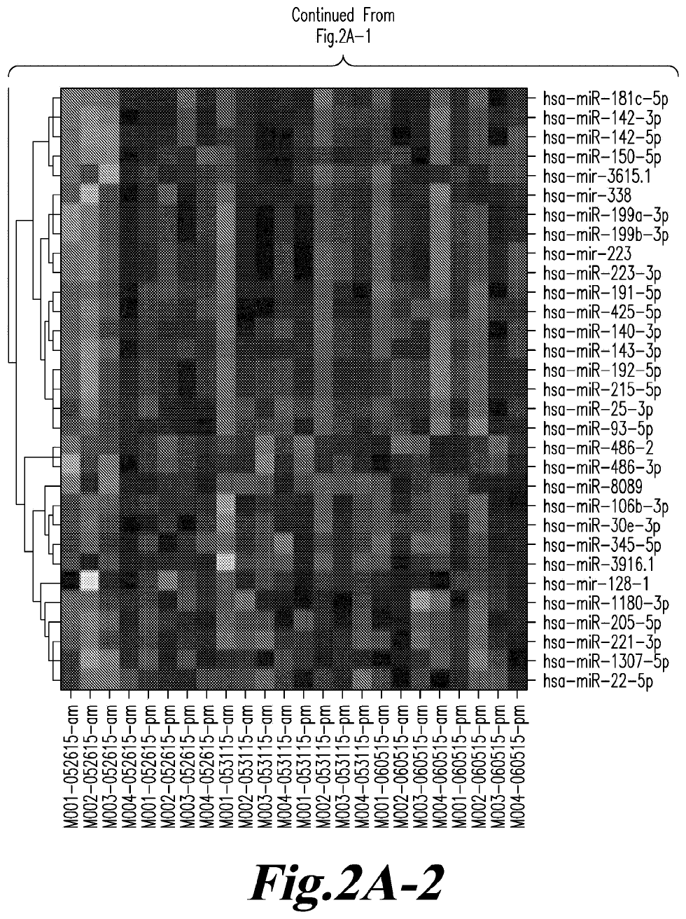

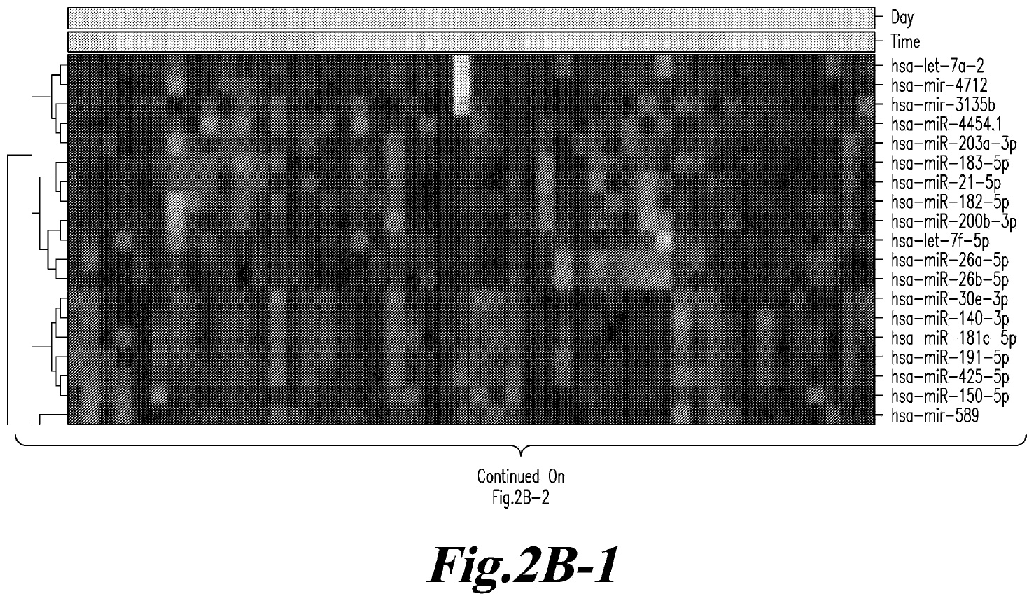

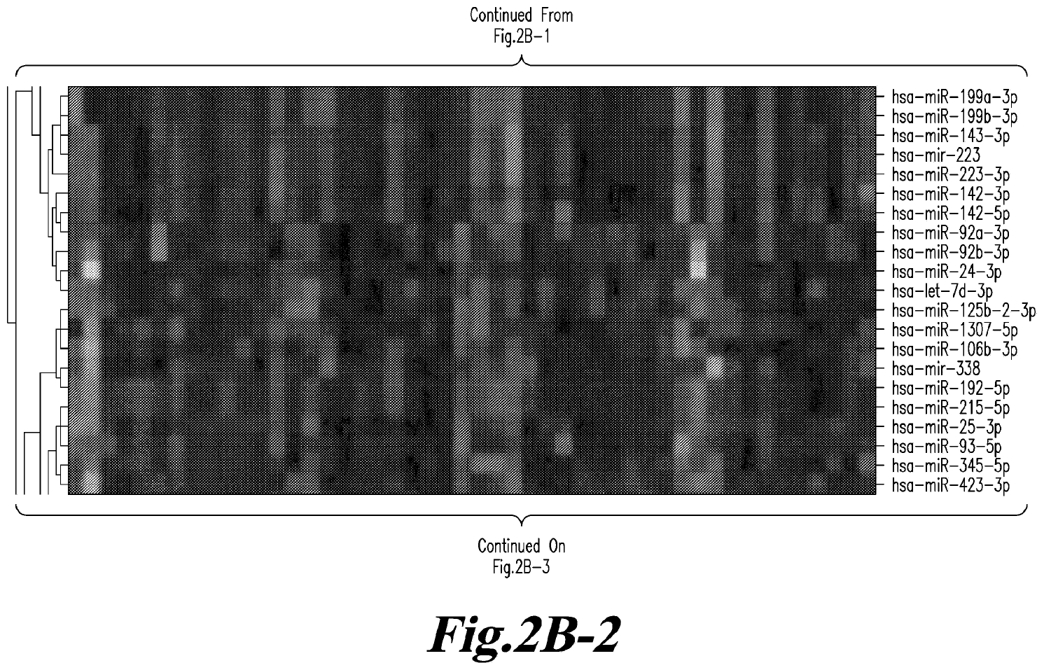

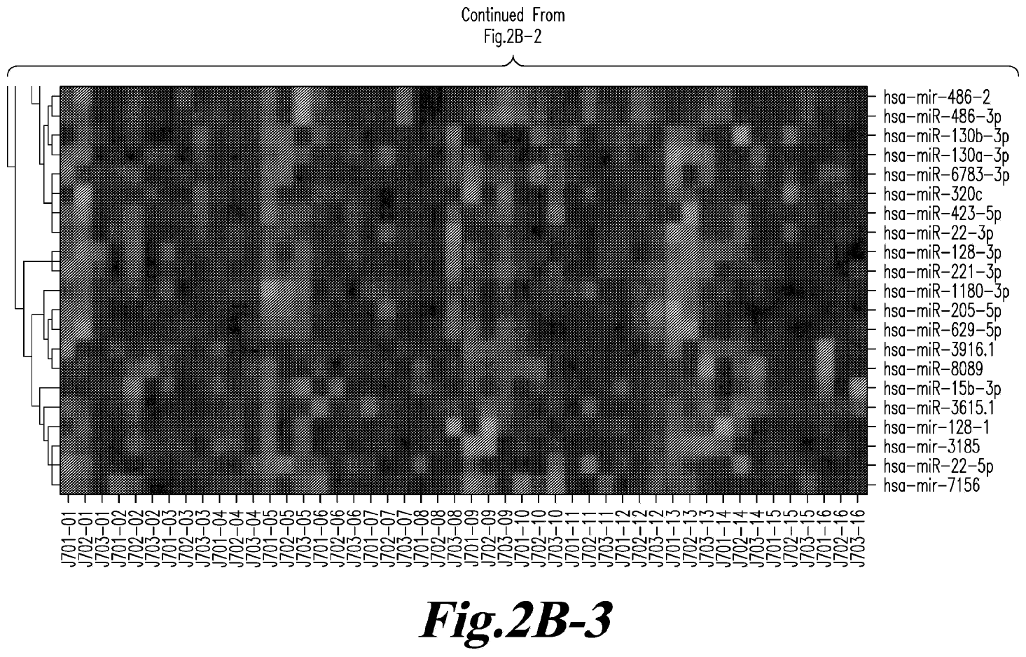

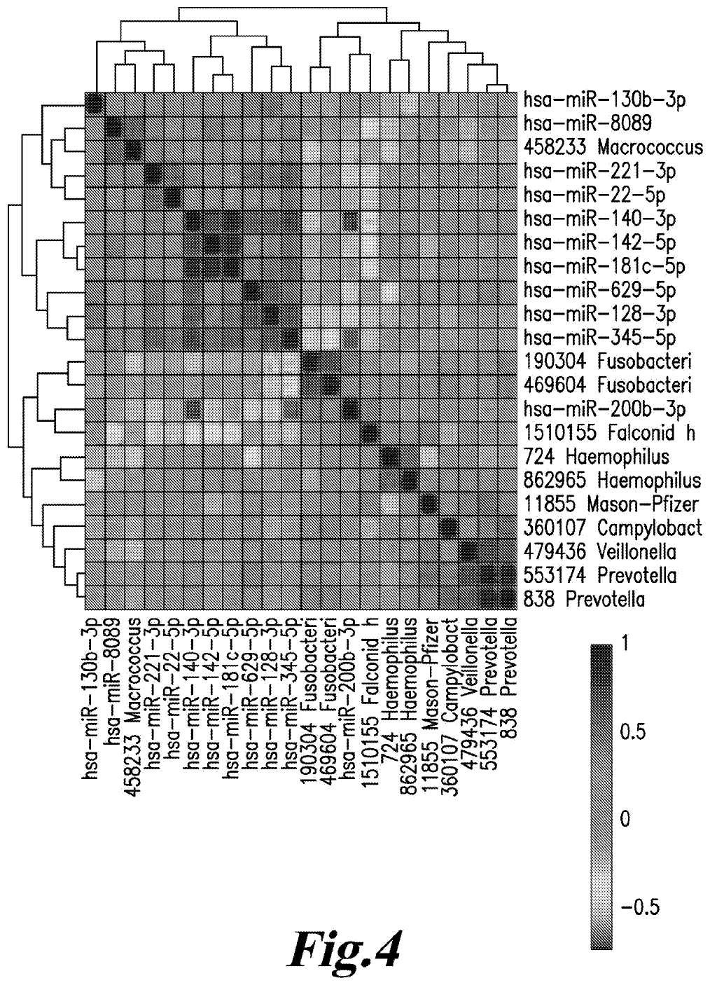

In some embodiments of this method an altered or abnormal concentration of one or more miRNAs is detected in at least one miRNAs selected from the group consisting of miR-142-5p, miR-130b-3p, miR-629-5p, miR-140-3p, miR-128-3p, miR-181c-5p, miR-345-5p, miR22-5p, miR-8089, miR-221-3p, and miR-200b-5p; from the group consisting of miR-629-5p, miR-24-3p, miR-200b-3p, miR-261-5p, miR-203a-3 p, miR-142-5p, miR-181c-5p, miR-26a-5p, miR-203a-3p, miR-24-3p, miR-22-5p, miR-142-5p, miR181c-5p and miR-181c-5p; from the group of miRNAs described by FIGS. 2A, 2B or 4; or from the group of miRNAs described elsewhere herein; or a mi-RNA having the same or similar seed as said miRNAs. In some embodiments of this method only miRNA concentrations will be determined, in other embodiments only levels of RNA expression of salivary microbes, and in still others both miRNA concentrations and levels of salivary microbe RNA expression.

In some embodiments, this method measures the level of RNA expression in one or more salivary microbes selected from the group consisting of Falconid herpesvirus, Prevotella melaninogenica ATCC 25845, Haemophilus parainfluenzae T3 T1, Veillonella parvula DSM 2008, Macrococcus caseolyticus JSCC5402, Fusobaterium nucleatum subsp. nucleatum 25586, Haemophilus, Fusobacterium nucleatum subsp. vincentii, Mason-Pfizer monkey virus, Camplyobacer hominis ATCC, and Prevotella; or from any other microbial RNAs described herein or in the Supplementary Tables; or a microbe having a genome that is at least 90, 95, 96, 97, 98, 99, 99.5 or 100% similar or identical thereto. BLASTN may be used to identify a polynucleotide sequence having at least 70%, 75%, 80%, 85%, 87.5%, 90%, 92.5%, 95%, 97.5%, 98%, 99% sequence identity to a reference polynucleotide. A representative BLASTN setting optimized to find highly similar sequences uses an Expect Threshold of 10 and a Wordsize of 28, max matches in query range of 0, match/mismatch scores of 1/−2, and linear gap cost. Low complexity regions may be filtered/masked. Default settings are described by and incorporated by reference to http://_blast.ncbi.nlm.nih.gov/Blast.cgi?PROGRAM=blastn&BLAST_PROGRAMS=megaBl ast&PAGE_TYPE=BlastSearch&SHOW_DEFAULTS=on&LINK_LOC=blasthome (last accessed Mar. 19, 2018)(incorporated by reference).

In certain embodiments of this method, the abnormal or altered miRNA concentration and/or expression level is associated with a disorder of gastrointestinal tract; associated with an eating disorder; associated with a gastric motility disorder; associated with a disorder of the nervous system; associated with a sexual dysfunction.; associated with a sleep disorder; associated with insomnia, apnea, or restless leg syndrome; associated with depression, anxiety, cognitive impairment, hyperactivity, anhedonia, dementia, amnesia or addiction; associated with a movement disorder; associated with a disorder of the glymphatic system; associated with a neurodegenerative disease; associated with a concussion, mTBI or TBI; associated with physical exertion or exercise; associated with a drug or other agent exogenous agent that affects temporal rhythm; associated with a microbe, hormone, or other endogenous agent that affects temporal rhythm; or associated with travel or exposure to light.

Another aspect of the invention is a composition having two or more primers or probes that detect miRNAs or microbial RNA expression levels that are associated with one or more abnormal or altered temporal rhythms, such as an altered diurnal or circadian rhythm. The composition may be in the form of a kit for detection of miRNAs or microbial RNA expression levels in saliva comprising 1, 2, 3, 4, 5, 6, 7, 8, 9, 10, 20, 50 or more probes that recognize miRNAs or microbial RNAs and optionally, excipients, buffers, platforms, containers, indicators, packing materials or instructions for use. The kit may further include at least one of the following: (a) one randomly generated miRNA sequence adapted to be used as a negative control; (b) at least one oligonucleotide sequence derived from a housekeeping gene, used as a standardized control for total RNA degradation; or (c) at least one randomly-generated sequence used as a positive control. Alternatively, a probe set may include miRNA probes having ribonucleotide sequences corresponding to DNA sequences from particular microbiomes described herein.

Such a composition or kit may be in the form of a microarray comprising a set of probes comprising nucleotide sequences capable of detecting and quantifying expression at least one miRNA sequence and/or microbial RNA sequence present in a saliva sample that correlates to an abnormal or altered temporal rhythm or dysrhythmia. The microarray may comprise a set of at least 2, 3, 4, 5, 6, 7, 8, 9, 10, 15, 20, 25, 30, 35, 40, 45, 50 or more primers or probes comprising nucleotide sequences that identify concentrations of miRNAs and/or microbial RNA expression levels that correlate to an abnormal or altered temporal rhythm or dysrhythmia.

Another aspect of the invention is a method of monitoring the progression of a disease, disorder or condition state associated with a temporal rhythm in a subject, the method comprising: analyzing at least two biological samples from the same subject taken at different time points to determine count and time-of-day normalized expression levels of one or more miRNAs or microbial RNAs in each of the at least two biological samples, and comparing the determined levels of the miRNAs or microbial RNAs over time to determine whether a subject's count and time-of-day normalized expression levels of one or more miRNAs is changing over time; wherein an increase or decrease in the count and time-of-day normalized expression levels of the one or more miRNAs or microbial RNAs over time is indicative of a progression of an abnormal or disrupted temporal rhythm, and a positive or negative difference in expression levels of the count and time-of-day normalized expression levels of the one or more miRNAs or microbial RNAs over time are indicative of the progression of symptoms of abnormal or disrupted temporal rhythm in the subject.

The invention also encompasses a method of comparing the epigenetic and/or microbiome data for a subject suspected to have an abnormal or altered temporal rhythm or dysrhythmia to one or more healthy control subjects or a compendium of healthy control subjects, each healthy control-subject being known not to have said an abnormal or altered temporal rhythm or dysrhythmia or have symptoms of said an abnormal or altered temporal rhythm or dysrhythmia, the method comprising determining the count of one or more miRNA and/or microbial RNA levels in a biological sample taken from a subject, normalizing the subject's epigenetic and/or microbiome genetic sequence data to account for inter-sample count variations; such count normalization utilizing one or more invariant miRNAs or microbial RNAs so as to represent data in proportion to their relative expression, or otherwise, determining the time of day that the biological sample was taken, applying a time-of-day normalization to the count normalized miRNAs and/or microbial RNAs by using the time-of-day to further normalize the subject's miRNA and/or microbial RNA expression levels relative to time-of-day, comparing the count and time-of-day normalized expression level(s) of the one or more miRNAs and/or microbial RNAs against the count and time-of-day normalized expression level(s) of one or more control miRNAs and/or one or more control microbial RNAs from one or more healthy control-subjects or a compendium of healthy control-subjects, wherein an increase or decrease in the expression level(s) of the one or more of the subject's miRNAs and/or microbial RNAs as compared to the same one or more miRNAs and/or microbial RNAs from one or more healthy control-subjects or a compendium of healthy control-subjects is indicative that the subject may have an abnormal or altered temporal rhythm or dysrhythmia.

In another embodiment, the invention involves a method of monitoring the progression or regression of an abnormal or altered temporal rhythm or dysrhythmia in a subject, the method comprising analyzing at least two biological samples, preferably saliva samples, from the same subject taken at different time points to determine the count and time-of-day normalized expression level of one or more miRNAs and/or microbial RNAs in each of the at least two biological samples, and comparing the determined level(s) of the one or more miRNAs and/or microbial RNAs over time to determine if the subject's count and time-of-day normalized expression level(s) of the one or more specific miRNAs and/or microbial RNAs is changing over time; wherein an increase or decrease in the count and time-of-day normalized expression level(s) of the one or more miRNAs and/or microbial RNAs over time may be indicative of a progression or regression of an abnormal or altered temporal rhythm or dysrhythmia or symptoms thereof in the subject, and the positive or negative difference in the expression level(s) of the count and time of day normalized expression level(s) of the one or more miRNAs and/or microbial RNAs over time may be indicative of the progression of an abnormal or altered temporal rhythm or dysrhythmia or symptoms thereof in the subject.

Another aspect of the invention is a forensic method comprising detecting a variation in a temporal rhythm comprising: detecting at least one abnormal or altered temporal pattern of miRNA or microbial RNA levels in saliva or other biological sample (including blood, plasma, serum, tears, sweat, urine, semen, mucosal secretions), compared to a control value from one or more normal subjects, and determining a time of death, time of bite or other injury, time of saliva or biological sample deposit, or other event based on a level of one or more miRNAs or microbial RNAs in saliva or other biological sample.

In another embodiment, the invention is directed to a method for assessing olfactory or gustatory senses or salivary gland status comprising detecting at least one abnormal or altered temporal pattern of miRNA or microbial RNA levels in saliva compared to a control value, and determining olfactory sense, gustatory sense, or salivary gland status based on a level of one or more miRNAs or microbial RNAs in saliva compared to a control; wherein said control may be a saliva sample from the same subject taken at a different time of day, a value from a subject having excellent olfactory or gustatory sensation or salivary gland status, or a value from a subject having an impaired olfactory or gustatory sensation or salivary gland status; and, optionally, selecting a subject having enhanced or diminished olfactory sense, gustatory sense or salivary status. This method may further encompass testing one or more olfactory sensations using a smell identification test or other test of olfactory sensation; testing one or more gustatory sensations with a taste test or other test of gustatory function; testing salivary gland status by a salivary gland function scan or other test of salivary function.

Another aspect of the invention is a method of normalizing epigenetic data to account for temporal variations in microRNA expression, the method comprising determining the count of one or more microRNAs (miRNAs) in a biological sample taken from a subject; normalizing the subject's epigenetic data to account for inter-sample count variations; such count normalization shall utilize one or more invariant miRNAs; determining the time of day that the biological sample was taken; and applying a time-of-day normalization to the count normalized miRNAs by using the time-of-day to further normalize the subject's miRNA expression levels relative to time-of-day.

The invention is also directed to method of comparing the epigenetic data for a subject with a suspected injury, disorder or disease state, which may include sleep disorders, to one or more healthy control-subjects or a compendium of healthy control subjects, each healthy control-subject being known not to have sustained the suspected injury, disorder or disease, the method comprising normalizing the subject's epigenetic data to account for count variations; further normalizing the epigenetic data to account for temporal variations in expression; comparing the count and time-of-day normalized expression level(s) of the one or more of the subject's miRNAs against the count and time-of-day normalized expression level(s) of the same one or more miRNAs from one or more healthy control-subjects or a compendium of healthy control-subjects; wherein an increase or decrease in the expression level(s) of the one or more of the subject's miRNAs against the same one or more miRNAs from one or more healthy control subjects or a compendium of healthy control-subjects is indicative that the subject may have sustained the suspected injury, disorder or disease state, inclusive of sleep disorders.

Another aspect of the invention is a method of monitoring the progression of an injury, disorder or disease state in a subject, the method comprising analyzing at least two biological samples from the subject taken at different time points to determine the count and time-of-day normalized expression level(s) of one or more specific miRNAs in each of the at least two biological samples; and comparing the determined level(s) of the one or more specific miRNAs over time to determine if the subject's count and time-of-day normalized expression level(s) of the one or more specific miRNAs is changing over time; wherein an increase or decrease in the count and time-of-day normalized expression level(s) of the one or more specific miRNAs over time may be indicative that the subject's injury, disorder or disease state, inclusive of sleep disorders, is improving or deteriorating.

The invention is also drawn to a method of normalizing epigenetic data to account for temporal variations in microbiome genetic sequence expression, the method comprising. determining the count of one or more microbiome (miBiome) genetic sequences, such as a total RNA expression level of a particular microorganism, in abiological sample taken from a subject; normalizing the subject's epigenetic data to account for inter-sample count variations; such count normalization may utilize one or more invariant RNAs or miRNAs; determining the time of day that the biological sample was taken; and applying a time-of-day normalization to the count normalized miRNAs by using the time-of-day to further normalize the subject's miRNA expression levels relative to time-of-day.

In another embodiment, the invention is directed to a method of comparing the epigenetic data for a subject with a suspected injury, disorder or disease state, which may include sleep disorders, to one or more healthy control-subjects or a compendium of healthy control subjects, each healthy control-subject being known not to have sustained the suspected injury, disorder or disease, the method comprising normalizing the subject's epigenetic data to account for count variations; further normalizing the epigenetic data to account for temporal variations in expression; comparing the count and time-of-day normalized expression level(s) of the one or more of the subject's miBiomes (microbial RNA) against the count and time-of-day normalized expression level(s) of the same one or more miBiomes from one or more healthy control-subjects or a compendium of healthy control-subjects, wherein an increase or decrease in the expression level(s) of the one or more of the subject's miBiomes against the same one or more miBiomes from one or more healthy control-subjects or a compendium of healthy control-subjects is indicative that the subject may have sustained the suspected injury, disorder or disease state, inclusive of sleep disorders.

Another embodiment of the invention is directed to a method of monitoring the progression of an injury, disorder or disease state, which may include sleep disorders, in a subject, the method comprising analyzing at least two biological samples from the subject taken at different time points to determine the count and time-of-day normalized expression level(s) of one or more specific miBiomes in each of the at least two biological samples, and comparing the determined level(s) of the one or more specific miBiomes over time to determine if the subject's count and time-of-day normalized expression level(s) of the one or more specific miRNAs is changing over time; wherein an increase or decrease in the count and time-of-day normalized expression level(s) of the one or more specific miBiomes over time may be indicative that the subject's injury, disorder or disease state, inclusive of sleep disorders, is improving or deteriorating.

Another aspect of the invention is method of detecting a miRNA and/or a miBiome sequence or a plurality of miRNAs and/or miBiome sequences in a first biological sample, comprising: obtaining a biological sample from a subject; creating a double-stranded, complementary DNA sequence (cDNA) for each of one or more miRNA or miBiome sequences selected from Group A circaMiRs, Group B circaMiRs and Group C miBiomes; and with real-time PCR or Next Generation Sequencing, Northern blotting or with microarrays, detecting the presence, absence or relative quantity of cDNAs, wherein the presence, absence or relative quantity of cDNA is indicative of the presence, absence or relative quantity of the complementary miRNA or miBiome sequence(s).

The invention is also directed to a method of detecting an miRNA and/or a miBiome sequence or a plurality of miRNAs and/or RNA expression level of miBiome sequences in a second biological sample, comprising: obtaining a biological sample from said subject at a second time point; creating a double-stranded, complementary DNA sequence (cDNA) for each of one or more miRNA or miBiome sequences selected from Group A circaMiRs, Group B circaMiRs and Group C miBiomes; and detecting with Northern Blot, realtime PCR or Next Generation Sequencing the presence, absence or relative quantity of cDNAs; wherein the presence, absence or relative quantity of cDNA in said biological sample from said second time point is indicative of the presence, absence or relative quantity of the complementary miRNA or miBiome sequence(s) at that second time point; and track the progression of any injury, disorder or disease, including sleep disorders, by comparing the results from the first time point to the results from the second time point.

### EXAMPLE

Subject assessment. This study was approved by the Institutional Review Board for the Protection of Human Subjects (IRB) at SUNY Upstate Medical University. Informed written consent was obtained for nine healthy human volunteers, and verbal assent was provided by all participating children.

Study design. A prospective cohort design employing high throughput RNA sequencing was used to examine salivary RNAs (human and microbial) for daily oscillations in concentration (FIG. 1). Nine healthy participants (3-55 years of age) were divided into three groups, and provided multiple saliva samples across a unique multi-day timeline (described below). Overlapping circadian RNA candidates from the first two independent sample sets were validated in a third sample set. Human miRNAs and microbial RNAs with confirmed diurnal variation were examined for associations in expression levels. Relationships between oscillating miRNAs and coding mRNA targets were also explored. Finally, the circadian RNA components were interrogated for functional relevance to human health and disease with the following three steps: 1) mRNA target networks for human miRNAs were identified in DIANA miRPath and Ingenuity Pathway Analyst (IPA, Qiagen), while metabolic pathways targeted by microbial RNAs were defined with MicrobiomAnalyst; 2) oscillating RNAs were retrospectively interrogated in a cohort of 140 children with autism spectrum disorder (ASD) with comorbid (n=77), or absent (n=63) sleep disturbance; and 3) the relationship of diurnal salivary RNAs with daily activities (tooth brushing, sleep, and eating) was assessed through Pearson correlation testing.

This study examined human miRNA and microbial RNA in saliva, because this biofluid provides on-demand access to repeated sampling of the GI tract at its sole point of entry, and represents a major site of host-environment interaction. Furthermore, studies of salivary miRNA in human patients have previously shown connections with brain-related dysfunction and potential relationships with time of collection [25, 26].

Participants. Participants included nine healthy volunteers, taking no daily medications, with no history of hospitalization, surgery, or sleep disorder. None of the participants had active dental caries. The nine participants were 3-55 years of age, 55% male, and 100% Caucasian. Participants provided saliva samples at various times of day on repeated days in four different sets of samples:

Sample Set 1: Morning and evening samples (n=24) collected at approximately 9 a.m. and 9 p.m. on days 1, 3, and 7 for 4 children (two male, two female; average age 7.5 yrs);

Sample Set 2: Early morning, early afternoon, late afternoon, and early evening samples n=48) collected at approximately 9 AM, 1:30 PM, 5:30 PM, and 9 p.m. on days 1, 5, 10 and 15 for three female children (average age 5.1 yrs), of whom two were part of Sample Set 1;

Sample Set 3: 12 samples collected at various times (ranging from 4 a.m. to midnight) on days 1 and 2 on two male children (average age 16.0 yrs) and their male and female parents (average age 51.5 yrs). Notably, detailed data regarding time of sleep, meals, and tooth brushing was collected for participants in Sample set 3.

Sample Set 4: Functional analysis of circadian RNAs was performed through retrospective analysis of data from an additional cohort of 140 children with ASD and comorbid sleep disturbance (n=77), or normal sleep (n=63). Salivary RNA was collected from these 140 children (2-6 years of age) at a single time-point, between 8 a.m. and 8 p.m. in a non-fasting state. ASD was confirmed through physician diagnosis, using the Diagnostic and Statistics Manual of the American Psychiatric Association, 5th Edition (DSM-5) criteria. Disordered sleep was identified through parent survey and chart review by research staff Participants with disordered sleep had either: 1) parent reported difficulty with sleep initiation or sleep maintenance; 2) ICD-10 diagnosis of disordered sleep (G47 or F51); or 3) a prescription for melatonin, clonidine, or mirtazapine with indication as a sleep aid. There was no difference in mean collection time between ASD subjects with (12:30 PM±2:48) and without (1:00 PM±3:00) disordered sleep (p=0.34). The sleep disorder group was 18% female (14/77) and had a mean age of 56 (±16) months. The non-sleep disorder group was 14% female (9/63) and had a mean age of 56 (±13) months.

Saliva collection and processing. Before collecting saliva samples, each subject rinsed their mouth with tap water. Approximately 1 mL of saliva was obtained through swab collection using an Oracollect RNA collection kit (DNA Genotek; Ottawa, Canada). Samples were stored at room temperature until processing. A Trizol method was used to purify the salivary RNA and a second round of purification was followed using an RNEasy mini column (Qiagen). Yield and quality of the RNA samples was assessed with the Agilent Bioanalyzer. This was done prior to library construction in accordance to the Illumina TruSeq Small RNA Sample Prep protocol (Illumina; San Diego, Calif.). Identification and quantification of saliva miRNA and microbial RNA was performed using next generation sequencing (NGS) on a NextSeq 500 instrument (Illumina), following the TruSeq Small RNA Library Preparation Kit protocol (Illumina, San Diego, Calif.). Alignment of mature miRNA reads was performed with the miRbase21 database using the Shrimp2 algorithm in Partek Flow software (Partek, Inc., St. Louis, Mo.). Mapping of unique microbial transcripts was performed using the K-Slam database, which references the NCBI Taxonomy database [27]. Taxons were defined by their family, genus, species, and subspecies (when available). The human miRNAs and microbial RNAs present in raw counts of 10 or more in at least 10% of samples were interrogated for oscillating expression. A quantile normalization technique was applied to the human miRNA and microbial RNA datasets separately, prior to statistical analysis.

Identification of oscillating salivary RNAs. A two-way analysis of variance (ANOVA) was performed using sample sets 1 and 2 based on binning the samples into their approximately replicated collection times, to identify host miRNAs and microbial RNAs that varied significantly (FDR<0.05) with collection time but not the day of collection (in order to eliminate RNAs which could be influenced by daily variations in routine). A subset of miRNAs and microbial RNAs that were highly associated with time of collection (R≥0.90 or 0.84 in sample sets 1 and 2, respectively; p<0.001) were then used in a naïve hold-out set (sample Set 3) to assess predictive accuracy for time of collection with a multivariate regression analysis. The miRNAs that showed the strongest circadian oscillations were termed CircaMiRs and the microbes that displayed the strongest oscillations in transcriptional activity were termed CircaMicrobes. Relationships between CircaMiRs and CircaMicrobes were investigated with a Pearson Correlation analysis. The Pearson Correlation Coefficient (PCC) is a measure of the linear correlation between two variables X and Y, giving a value between +1 and −1 inclusive, where 1 is total positive correlation, 0 is no correlation, and −1 is total negative correlation.

Functional Interrogation of CircaMiRs and CircaMicrobes. Classification of the mapped microbial RNAs within defined metabolic and functional categories was performed through conversion of microbial reads to Kyoto Encyclopedia of Genes and Genomes (KEGG) Orthology identifiers (KO IDs) which were mapped by MicrobiomeAnalyst software[28] (to a set of 202 different KEGG Modules, KEGG Pathways, and COG Categories. For each participant, KEGG and COG data were summed across four collection periods (i.e. 7-9 AM, 10 AM-2 PM, 3-6 PM, 7-10 PM) for all of the days saliva samples were collected. Changes in expression of individual functional clusters were explored with a non-parametric Kruskal Wallis ANOVA. Patterns in functional clusters across the four time periods were visualized with hierarchical clustering analysis and a partial least squares discriminant analysis in MetaboAnalyst software.

The potential biologic impact of the CircaMiRs was investigated through functional annotation of their high confidence mRNA targets (p<0.05, Micro-T Score>0.95) in DIANA miRPath v3 software and Ingenuity Pathway Analyst software (IPA, Qiagen). KEGG pathways over-represented by these mRNA targets were determined with Fisher's Exact test with FDR correction (FDR<0.05). Inter-relatedness of protein products for the mRNA targets was explored in String v10.5. Alignment of salivary RNA to the RefSeq Transcripts database in Partek Flow permitted quantification of local (oropharyngeal) mRNA targets for salivary CircaMiRs (that were ≤50 base pairs). Relationships between CircaMiRs and mRNA targets were explored with Pearson's correlations.

To further explore the potential biological significance of the miRNA data, we examined the levels of the oscillating salivary CircaMiRs in the same cohort of 2-6 year old children with ASD examined for miRNA expression who either had normal sleep patterns (n=63) or disordered sleep symptoms (n=77). Group differences in mean salivary CircaMiR expression between the sleep disorder and non-sleep disorder groups were identified with a non-parametric Mann Whitney U-test. A two-way ANOVA assessed relationships between CircaMiRs, disordered sleep, and collection time, as well as sleep disorder-time interactions. Finally, a multivariate linear regression was used to determine the ability of the most robust CircaMiRs to predict collection time in the ASD children with and without sleep disorders.

Influence of daily routines on the oral transcriptome. To investigate the potential impact of daily routines on salivary miRNA and microbial RNA levels we examined associations between the oral transcriptome in sample Set 3 and Set 1) time since last meal (in hours); 2) time since last tooth brushing (in hours); and 3) time since last sleep (in hours). Significant relationships (|R|>0.40; FDR<0.05) between these three variables and salivary RNA levels were reported.

Salivary miRNA analysis. An overview of the sample sets and analyses is provided (FIG. 1).

Sample Set 1 contained 24 saliva samples collected at 2 time-points (˜9 AM, 9 PM) on 3 days from 4 participants. There were a total of 98 miRNAs in Set 1 with a significant effect of collection time (FDR<0.01) and no effect of day of collection (FDR>0.05).

Sample Set 2 contained 48 samples collected at 4 time-points (˜9 a.m., 1:30 p.m., 5:30 p.m., 9 p.m.) on 4 days from 3 participants. There were a total of 123 miRNAs in Set 2 that showed a significant effect of collection time and no effect of day. Levels of 61 miRNAs were similarly affected by time of collection in both sample sets and were defined as putative CircaMiRs See Supplementary Table 1.

Hierarchical (heat map) clustering using salivary concentrations of the 61 CircaMiRs was performed for sample Set 1 (FIG. 2A) and sample Set 2 (FIG. 2B). In both sample sets, the majority of CircaMiRs (n=49; 80%) demonstrated lower levels in the morning and higher levels in the evening. Examination of the 61 miRNAs across four time points (sample Set 2) revealed only a single oscillation (i.e. a single daily peak) between 9 a.m. and 9 p.m. These daily oscillations were consistent across days of collection and across participants, as reflected by the lack of significant day effects in the two-way ANOVA. See Supplementary Table 1.

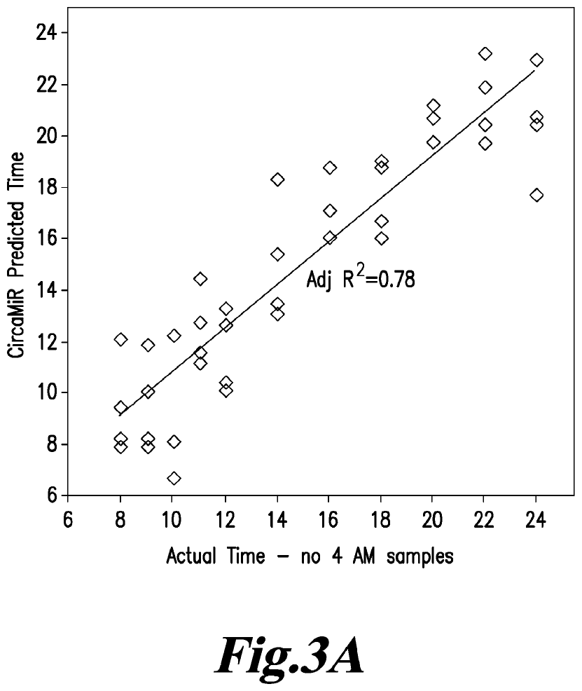

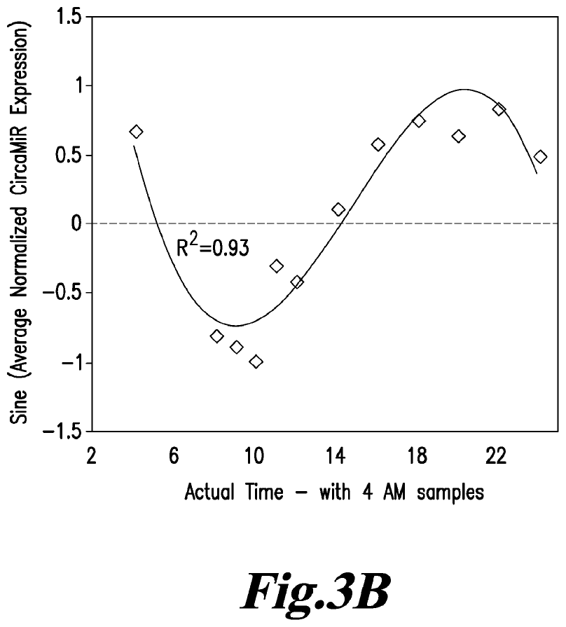

From the 61 CircaMiR candidates, 11 miRNAs were identified as robust multivariate predictors of collection time through a feature selection algorithm using a linear regression analysis. The regression model accurately predicted collection time in all 3 sample sets, with Multiple R values ranging from 0.805-0.956 and Adjusted R2 values ranging from 0.54-0.833 (Table 1, upper). Notably, the multivariate model performed best when applied to samples collected during a wakeful state (9 a.m.-12 p.m.) and model performance significantly improved in sample Set 3 when 4 a.m. samples were excluded (Adjusted R2=0.880 vs 0.794, Table 1, upper). This improvement was due to non-linear trends in the expression data during the overnight period (a circadian oscillation of high values back to low values and vice versa). In fact, the predictive utility of the linear regression model (R2=0.79; FIG. 3A) was even found to be inferior to a non-linear regression model that used the sine-transformed average miRNA values for just one of the 61 CircaMiRs in the Set 3 samples (R2=0.93; FIG. 3B). Interestingly, further inspection of the alpha (intercept) and beta (slope) coefficient terms across the independent sample set regressions indicated a very high degree of internal consistency in these models (Table 1, lower), with highly significant correlations present between all sets of model term comparisons except sample Set 1 and sample Set 3 with the 4 a.m. samples included.

Table 1. Salivary miRNA and microbial RNA model performance for predicting collection time.

TABLE 1
Salivary miRNA and microbial RNA model performance
for predicting collection time.

| | | | | Mean |
|---|---|---|---|---|
| | | | | Absolute |
| | Multiple R | Adjusted R² | P-value | Error (%) |
| microRNAs | | | | |
| Sample set 1 (n = 24) | 0.956 | 0.833 | 9.5E−05 | 9.2 |
| Sample set 2 (n = 48) | 0.805 | 0.540 | 1.8E−05 | 14.6 |
| Sample set 3 (n = 48) | 0.918 | 0.794 | 2.8E−11 | 12.7 |
| Sample set 3 (no 4AM, n = 44) | 0.954 | 0.880 | 1.1E−13 | 8.1 |
| microbial RNAs | | | | |
| Sample set 1 (n = 24) | 0.927 | 0.732 | 0.0013 | 13.1 |
| Sample set 2 (n = 48) | 0.784 | 0.496 | 7.5E−05 | 15.0 |
| Sample set 3 (n = 48) | 0.770 | 0.468 | 1.8E−04 | 21.4 |
| Sample set 3 (no 4AM, n = 44) | 0.849 | 0.624 | 3.6E−06 | 15.1 |

| | | | |
|---|---|---|---|
| Set 1 | Set2 | Set 3 | Set 3 no 4 am |

Correlations of miRNA model terms (11 beta coefficients + intercept)

| | | | | |
|---|---|---|---|---|
| Set 1 | | 0.7469 | 0.5399 | 0.7207 |
| Set 2 | 0.0053 | | 0.7060 | 0.8409 |
| Set 3 | 0.0698 | 0.0103 | | 0.9647 |
| Set 3 no 4 am | 0.0082 | 0.0006 | <.0001 | |

Correlations of microbial RNA model terms (11 beta coefficients + intercept)

| | | | | |
|---|---|---|---|---|
| Set 1 | | 0.8929 | 0.8319 | 0.9066 |
| Set 2 | <.0001 | | 0.8699 | 0.9542 |
| Set 3 | 0.0008 | 0.0002 | | 0.9630 |
| Set 3 no 4 am | <.0001 | <.0001 | <.0001 | |

SUPPLEMENTARY TABLE 1
Circadian miRNAs in Sample Sets 1 and 2 Identified by Two-Way ANOVA

| | Sample Set 1 | | | | Sample Set |
|---|---|---|---|---|---|
| MicroRNA | Day | Time | Interaction | MicroRNA | Day |
| hsa-miR-3135b | 0.99745 | 2.03E−06 | 0.91865 | hsa-miR-423-5p | 0.094015 |
| hsa-miR-598-5p | 0.99745 | 7.70E−06 | 0.91865 | hsa-mir-3185 | 0.094015 |
| hsa-miR-620 | 0.99745 | 1.82E−05 | 0.91865 | hsa-miR-3916 | 0.11977 |
| hsa-mir-1268a | 0.99745 | 1.82E−05 | 0.91865 | hsa-let-7f-2-3p | 0.14444 |
| hsa-miR-92a-3p | 0.99745 | 1.82E−05 | 0.91865 | hsa-let-7c-5p | 0.15484 |
| hsa-mir-3135b | 0.99745 | 1.82E−05 | 0.93292 | hsa-miR-21-5p | 0.15872 |
| hsa-miR-26a-5p | 0.99745 | 4.47E−05 | 0.91865 | hsa-miR-130a-3p | 0.1778 |
| hsa-miR-30e-5p | 0.99745 | 5.48E−05 | 0.91865 | hsa-miR-6783-3p | 0.19511 |
| hsa-miR-374a-3p | 0.99745 | 6.97E−05 | 0.91865 | hsa-miR-320a | 0.19511 |
| hsa-miR-143-3p | 0.99745 | 0.009143 | 0.91865 | hsa-miR-8089 | 0.20869 |
| hsa-mir-223 | 0.99745 | 0.000154 | 0.91865 | hsa-miR-1277-5p | 0.23083 |
| hsa-miR-200b-3p | 0.99745 | 0.090181 | 0.91865 | hsa-miR-125b-2-3p | 0.24161 |
| hsa-miR-221-3p | 0.99745 | 0.000181 | 0.91865 | hsa-miR-130b-3p | 0.25896 |
| hsa-mir-26a-2 | 0.99745 | 0.090181 | 0.92592 | hsa-miR-26b-5p | 0.3333 |
| hsa-miR-223-3p | 0.99745 | 0.000209 | 0.91865 | hsa-miR-24-3p | 0.33337 |
| hsa-miR-182-5p | 0.99745 | 0.000232 | 0.91865 | hsa-miR-3607-5p | 0.33337 |
| hsa-rmR-183-5p | 0.99745 | 0.000418 | 0.91865 | hsa-miR-4642 | 0.33828 |
| hsa-miR-378d | 0.99745 | 0.000418 | 0.91865 | hsa-mir-3615.1 | 0.34645 |
| hsa-mir-4289 | 0.99745 | 0.000418 | 0.91865 | hsa-miR-3613-5p | 0.36794 |
| hsa-mir-1248 | 0.99745 | 0.000461 | 0.91865 | hsa-miR-15b-3p | 0.36869 |
| hsa-miR-378a-5p | 0.99745 | 0.000561 | 0.91865 | hsa-let-7d-3p | 0.36869 |
| hsa-miR-152-3p | 0.99745 | 0.000562 | 0.91865 | hsa-mir-3615 | 0.36869 |
| hsa-mir-6131 | 0.99745 | 0.000619 | 0.99632 | hsa-miR-15a-5p | 0.36869 |
| hsa-miR-4436b-3p | 0.99745 | 0.001251 | 0.91865 | hsa-miR-320c | 0.36869 |
| hsa-miR-21-5p | 0.99745 | 0.001413 | 0.91865 | hsa-miR-340-5p | 0.3763 |
| hsa-mir-598 | 0.99745 | 0.001795 | 0.91865 | hsa-miR-3135b | 0.3763 |
| hsa-miR-365a-3p | 0.99745 | 0.001795 | 0.91865 | hsa-miR-574-3p | 0.3871 |
| hsa-miR-365b-3p | 0.99745 | 0.001795 | 0.91865 | hsa-miR-423-3p | 0.39521 |
| hsa-miR-199b-3p | 0.99745 | 0.001795 | 0.95384 | hsa-miR-203a-3p | 0.41763 |
| hsa-miR-199a-3p | 0.99745 | 0.001795 | 0.95384 | hsa-miR-142-5p | 0.41763 |
| hsa-miR-193b-3p | 0.99745 | 0.001872 | 0.91865 | hsa-miR-22-5p | 0.45214 |
| hsa-miR-6724-5p | 0.99745 | 0.002765 | 0.91865 | hsa-miR-142-3p | 0.45262 |
| hsa-miR-375 | 0.99745 | 0.002765 | 0.93292 | hsa-miR-625-3p | 0.47762 |
| hsa-mir-3185 | 0.99745 | 0.00359 | 0.91865 | hsa-miR-96-5p | 0.48854 |
| hsa-miR-191-5p | 0.99745 | 0.00365 | 0.91865 | hsa-miR-4454.1 | 0.48997 |
| hsa-mir-4438 | 0.99745 | 0.003671 | 0.91865 | hsa-miR-200b-3p | 0.48997 |
| hsa-mir-183 | 0.99745 | 0.003671 | 0.95285 | hsa-miR-8059 | 0.4947 |
| hsa-miR-4794 | 0.99745 | 0.003746 | 0.91865 | hsa-miR-182-5p | 0.49509 |
| hsa-mir-5697 | 0.99745 | 0.00378 | 0.91865 | hsa-mir-145 | 0.53333 |
| hsa-miR-95-3p | 0.99745 | 0.003841 | 0.92021 | hsa-miR-374c-5p | 0.56909 |
| hsa-miR-27a-5p | 0.99745 | 0.00397 | 0.91865 | hsa-mir-192 | 0.58075 |
| hsa-mir-6844 | 0.99745 | 0.004273 | 0.92584 | hsa-miR-181c-5p | 0.60007 |
| hsa-miR-429 | 0.99745 | 0.004348 | 0.91865 | hsa-mir-3613 | 0.60859 |
| 2. | | | | | |
| hsa-miR-532-5p | 0.99745 | 0.00454 | 0.92592 | hsa-miR-6739-5p | 0.61755 |
| hsa-miR-23b-3p | 0.99745 | 0.005148 | 0.91865 | hsa-mir-3908 | 0.61755 |
| hsa-miR-320a | 0.99745 | 0.005148 | 0.98489 | hsa-miR-128-3p | 0.62521 |
| hsa-mir-7110 | 0.99745 | 0.005348 | 0.91865 | hsa-let-7f-5p | 0.62521 |
| hsa-miR-142-3p | 0.99745 | 0.005348 | 0.91865 | hsa-miR-6854-5p | 0.62521 |
| hsa-mir-4454 | 0.99745 | 0.005431 | 0.92605 | hsa-miR-183-5p | 0.62521 |
| hsa-miR-26b-5p | 0.99745 | 0.005976 | 0.91865 | hsa-mir-7156 | 0.62521 |
| hsa-miR-548ah-5p | 0.99745 | 0.006108 | 0.92176 | hsa-miR-92a-3p | 0.62521 |
| hsa-miR-205-5p | 0.99745 | 0.006278 | 0.91865 | hsa-miR-143-3p | 0.62521 |
| hsa-mir-4712 | 0.99745 | 0.006278 | 0.95447 | hsa-miR-26a-5p | 0.62521 |
| hsa-miR-2110 | 0.99745 | 0.006687 | 0.91865 | hsa-miR-150-5p | 0.62521 |
| hsa-miR-1255a | 0.99745 | 0.00695 | 0.91865 | hsa-miR-16-5p | 0.62521 |
| hsa-miR-590-3p | 0.99745 | 0.00695 | 0.91865 | hsa-mir-338 | 0.62521 |
| hsa-let-7a-2 | 0.99745 | 0.006952 | 0.94513 | hsa-mir-589 | 0.62521 |
| hsa-miR-548ad-5p | 0.99745 | 0.007017 | 0.91865 | hsa-miR-1307-5p | 0.62521 |
| hsa-miR-24-3p | 0.99745 | 0.007668 | 0.91865 | hsa-mir-450b | 0.62521 |
| hsa-let-7f-5p | 0.99745 | 0.008242 | 0.91865 | hsa-mir-4712 | 0.62521 |
| hsa-mir-374a | 0.99745 | 0.008352 | 0.92595 | hsa-mir-486-2 | 0.62726 |
| hsa-miR-4457 | 0.99745 | 0.008363 | 0.92592 | hsa-miR-92b-3p | 0.6291 |
| hsa-mir-6841 | 0.99745 | 0.009184 | 0.94798 | hsa-miR-22-3p | 0.64895 |
| hsa-miR-6826-5p | 0.99745 | 0.009855 | 0.91865 | hsa-miR-205-5p | 0.65209 |
| hsa-mir-1915 | 0.99745 | 0.010113 | 0.91865 | hsa-miR-375 | 0.69188 |
| hsa-miR-1255b-5p | 0.99745 | 0.010317 | 0.91865 | hsa-miR-345-5p | 0.6946 |
| hsa-miR-3915 | 0.99745 | 0.010317 | 0.91865 | hsa-miR-3202 | 0.71649 |
| hsa-miR-4454 | 0.99745 | 0.010404 | 0.91865 | hsa-let-7f-2 | 0.71649 |
| hsa-mir-708 | 0.99745 | 0.010404 | 0.91865 | hsa-miR-93-5p | 0.72367 |
| hsa-mir-338 | 0.99745 | 0.010404 | 0.91865 | hsa-miR-25-3p | 0.73103 |
| hsa-miR-320b | 0.99745 | 0.011606 | 0.96841 | hsa-mir-223 | 0.73665 |
| hsa-miR-200a-3p | 0.99745 | 0.011642 | 0.91865 | hsa-miR-338-3p | 0.7378 |
| hsa-mir-548i-3 | 0.99745 | 0.011849 | 0.91865 | hsa-miR-223-3p | 0.74112 |
| hsa-let-7a-1 | 0.99745 | 0.013042 | 0.91865 | hsa-miR-629-5p | 0.74693 |
| hsa-miR-125b-2-3p | 0.99745 | 0.013555 | 0.91865 | hsa-mir-2355 | 0.74693 |
| hsa-miR-6770-5p | 0.99745 | 0.013883 | 0.91865 | hsa-mir-7-1 | 0.75072 |
| hsa-miR-345-5p | 0.99745 | 0.014099 | 0.91865 | hsa-let-7a-2 | 0.75631 |
| hsa-mir-429 | 0.99745 | 0.014099 | 0.94181 | hsa-miR-619-5p | 0.79015 |
| hsa-mir-3065 | 0.99745 | 0.014208 | 0.91865 | hsa-miR-199a-3p | 0.7969 |
| hsa-miR-19b-3p | 0.99745 | 0.014208 | 0.92176 | hsa-miR-199b-3p | 0.7969 |
| hsa-miR-221-5p | 0.99745 | 0.015905 | 0.92176 | hsa-miR-140-3p | 0.80665 |
| hsa-miR-1273g-3p | 0.99745 | 0.016087 | 0.91865 | hsa-mir-7851 | 0.82665 |
| hsa-miR-140-3p | 0.99745 | 0.016841 | 0.97161 | hsa-miR-98-5p | 0.83581 |
| hsa-miR-130a-3p | 0.99745 | 0.018181 | 0.91865 | hsa-miR-191-5p | 0.86273 |
| hsa-let-7d-3p | 0.99745 | 0.01915 | 0.91865 | hsa-miR-425-5p | 0.86624 |
| hsa-mir-34b | 0.99745 | 0.01915 | 0.91865 | hsa-mir-128-1 | 0.8719 |
| hsa-mir-382 | 0.99745 | 0.01915 | 0.91865 | hsa-mir-4286 | 0.8719 |
| hsa-mir-3199-2 | 0.99745 | 0.01915 | 0.91865 | hsa-miR-221-3p | 0.87442 |
| hsa-miR-128-3p | 0.99745 | 0.019367 | 0.91865 | hsa-miR-222-3p | 0.92545 |
| hsa-miR-24-2-5p | 0.99745 | 0.019367 | 0.91865 | hsa-miR-106b-3p | 0.93266 |
| hsa-miR-96-5p | 0.99745 | 0.020138 | 0.91865 | hsa-miR-30e-3p | 0.95082 |
| hsa-miR-215-5p | 0.99745 | 0.021359 | 0.93292 | hsa-miR-192-5p | 0.97373 |
| hsa-miR-192-5p | 0.99745 | 0.021359 | 0.93292 | hsa-miR-215-5p | 0.97373 |
| hsa-miR-486-5p | 0.99745 | 0.021359 | 0.95547 | hsa-miR-1180-3p | 0.97373 |
| hsa-mir-6499 | 0.99745 | 0.02389 | 0.98202 | hsa-miR-3916.1 | 0.98862 |
| hsa-mir-4649 | 0.99745 | 0.024055 | 0.91865 | hsa-miR-486-3p | 0.99238 |
| hsa-mir-6883 | 0.99745 | 0.024055 | 0.91865 | hsa-miR-708-5p | 0.99688 |
| hsa-miR-574-3p | 0.99745 | 0.024055 | 0.93292 | | |
| hsa-miR-378f | 0.99745 | 0.024055 | 0.93501 | | |
| hsa-mir-549a | 0.99745 | 0.02457 | 0.91865 | | |
| hsa-miR-423-5p | 0.99745 | 0.025226 | 0.91865 | | |
| hsa-miR-142-5p | 0.99745 | 0.025456 | 0.97978 | | |
| hsa-miR-374c-3p | 0.99745 | 0.025748 | 0.91865 | | |
| hsa-miR-5001-3p | 0.99745 | 0.025748 | 0.92719 | | |
| hsa-mir-1273g | 0.99745 | 0.026194 | 0.91865 | | |
| hsa-miR-1266-5p | 0.99745 | 0.028529 | 0.91865 | | |
| hsa-miR-1293 | 0.99745 | 0.031717 | 0.91865 | | |
| hsa-mir-664a | 0.99745 | 0.032183 | 0.92021 | | |
| hsa-mir-4768 | 0.99745 | 0.03458 | 0.91865 | | |
| hsa-miR-32p-5p | 0.99745 | 0.035832 | 0.91865 | | |
| hsa-mir-5588 | 0.99745 | 0.035955 | 0.91865 | | |
| hsa-mir-4762 | 0.99745 | 0.036049 | 0.91865 | | |
| hsa-miR-8058 | 0.99745 | 0.036049 | 0.92592 | | |
| hsa-miR-5697 | 0.99745 | 0.038766 | 0.91865 | | |
| hsa-mir-486-2 | 0.99745 | 0.038766 | 0.91865 | | |
| hsa-mir-126 | 0.99745 | 0.038766 | 0.97415 | | |
| hsa-mir-31 | 0.99745 | 0.038766 | 0.97759 | | |
| hsa-miR-203b-3p | 0.99745 | 0.038766 | 0.97887 | | |
| hsa-mir-6133 | 0.99745 | 0.040296 | 0.91865 | | |
| hsa-mir-621 | 0.99745 | 0.040296 | 0.91865 | | |
| hsa-mir-6510 | 0.99745 | 0.041301 | 0.91865 | | |
| hsa-mir-96 | 0.99745 | 0.041301 | 0.99807 | | |
| hsa-mir-365a | 0.99745 | 0.043207 | 0.91865 | | |
| hsa-miR-573 | 0.99745 | 0.043521 | 0.97415 | | |
| hsa-miR-181c-5p | 0.99745 | 0.043753 | 0.92176 | | |
| hsa-miR-148b-3p | 0.99745 | 0.045446 | 0.91865 | | |
| hsa-miR-135a-5p | 0.99745 | 0.046053 | 0.91865 | | |
| hsa-miR-4461 | 0.99745 | 0.646053 | 0.91865 | | |
| hsa-mir-4510 | 0.99745 | 0.046213 | 0.92176 | | |
| hsa-miR-29b-3p | 0.99745 | 0.047715 | 0.91865 | | |
| hsa-let-7a-5p | 0.99745 | 0.047715 | 0.91865 | | |
| hsa-mir-3665 | 0.99745 | 0.047715 | 0.91865 | | |
| hsa-mir-345 | 0.99745 | 0.047715 | 0.91865 | | |
| hsa-miR-98-5p | 0.99745 | 0.047715 | 0.91865 | | |
| hsa-miR-210-5p | 0.99745 | 0.047715 | 0.93431 | | |
| hsa-mir-5100 | 0.99745 | 0.047715 | 0.93744 | | |
| hsa-mir-6087 | 0.99745 | 0.048124 | 0.91865 | | |

*p-values for Day, Time, and Day-Time Interaction on 2-way ANOVA are shown

Salivary microbiome analysis. Sample Set 1 contained a total of 82 microbial RNAs with a significant effect of collection time (FDR<0.01) and no effect of day of collection (FDR>0.05). Sample Set 2 contained a total of 37 microbial RNAs with a significant effect of collection time and no effect of day of collection. Eleven microbial RNAs with diurnal oscillations in sample Sets 1 and 2 overlapped (Table 2). The 11 RNAs from these 11 distinct microbial species were defined as putative CircaMicrobes, and examined for their ability to predict collection time in sample Set 3. Table 2A—Group A and Group B miRNAs and Table 2B: Group C microorganisms appear on the next pages.

TABLE 2A
Group A and Group B circaMiRs

| | Group A circaMIRs | Group B circaMIRs |
|---|---|---|
| 1 | hsa-miR-106b-3p | hsa-let-7a-5p |
| 2 | hsa-miR-128-3p | hsa-let-7d-3p |
| 3 | hsa-miR-130a-3p | hsa-miR-101-3p |
| 4 | hsa-miR-15a-5p | hsa-miR-l0b-5p |
| 5 | hsa-miR-192-5p | hsa-miR-125b-2-3p |
| 6 | hsa-miR-199a-3p | hsa-miR-1307-5p |
| 7 | hsa-miR-199b-3p | hsa-miR-140-3p |
| 8 | hsa-miR-203a-3p | hsa-miR-142-3p |
| 9 | hsa-miR-221-3p | hsa-miR-143-3p |
| 10 | hsa-miR-26a-5p | hsa-miR-148b-3p |
| 11 | hsa-miR-26b-5p | hsa-miR-16-5p |
| 12 | hsa-miR-3074-5p | hsa-miR-181a-5p |
| 13 | hsa-miR-30e-3p | hsa-miR-181c-5p |
| 14 | hsa-miR-320a | hsa-miR-186-5p |
| 15 | hsa-miR-345-5p | hsa-miR-191-5p |
| 16 | hsa-miR-375 | hsa-miR-193a-5p |
| 17 | hsa-miR-423-3p | hsa-miR-200b-3p |
| 18 | hsa-miR-92a-3p | hsa-miR-205-5p |
| 19 | hsa-miR-93-5p | hsa-miR-215-5p |
| 20 | | hsa-miR-21-5p |
| 21 | | hsa-miR-223-3p |
| 22 | | hsa-miR-22-3p |
| 23 | | hsa-miR-23a-3p |
| 24 | | hsa-miR-23b-3p |
| 25 | | hsa-miR-24-3p |
| 36 | | hsa-miR-25-3p |
| 27 | | hsa-miR-29a-3p |
| 28 | | hsa-miR-30d-5p |
| 29 | | hsa-miR-320b |
| 30 | | hsa-miR-361-5p |
| 31 | | hsa-miR-363-3p |
| 32 | | hsa-miR-374a-3p |
| 33 | | hsa-miR-423-5p |
| 34 | | hsa-miR-425-5p |
| 35 | | hsa-miR-532-5p |
| 36 | | hsa-miR-574-3p |
| 37 | | hsa-miR-629-5p |
| 38 | | hsa-miR-8-98-5p |

TABLE 2B
Group C microorganisms (further information about these microbes may be accessed at
https://_ jgi.doe.gov/ or at http://_www.uniprot.org/proteomes/ both of which are
incorporated by reference).

| | | Sample set 1 | | | Sample set 2 | | |
|---|---|---|---|---|---|---|---|
| Taxon ID | Taxon Name | Day | Time | Interaction | Day | Time | Interaction |
| 1510155 | Falconid | 0.7246 | 0.0003 | 0.1104 | 0.9999 | 0.0009 | 0.9982 |
| | herpesvirus 1 | | | | | | |
| 553174 | Prevotella | 0.8213 | 0.0011 | 0.1693 | 0.9999 | 0.0359 | 0.9982 |
| | melaninogenica | | | | | | |
| | ATCC 25845 | | | | | | |
| 862965 | Haemophilus | 0.2276 | 0.0061 | 0.2426 | 0.9999 | 0.0045 | 0.9982 |
| | parainfluenzae | | | | | | |
| | T3T1 | | | | | | |
| 479436 | Veillonella | 0.7246 | 0.0076 | 0.1069 | 0.9999 | 0.0001 | 0.9982 |
| | parvula DSM | | | | | | |
| | 2008 | | | | | | |
| 458233 | Macrococcus | 0.0830 | 0.0338 | 0.1302 | 0.9999 | 0.0381 | 0.9982 |
| | caseolyticus | | | | | | |
| | JCSC5402 | | | | | | |
| 190304 | Fusobacterium | 0.9782 | 0.0127 | 0.1069 | 0.9999 | 0.0350 | 0.9982 |
| | nucleatum | | | | | | |
| | subsp. | | | | | | |
| | nucleatum | | | | | | |
| | ATCC 25586 | | | | | | |
| 724 | Haemophilus | 0.5928 | 0.0127 | 0.2426 | 0.9999 | 0.0139 | 0.9982 |
| 469604 | Fusobacterium | 0.7246 | 0.0209 | 0.1069 | 0.9999 | 0.0187 | 0.9982 |
| | nucleatum | | | | | | |
| | subsp. vincentii | | | | | | |
| | 3136A2 | | | | | | |
| 11855 | Mason-Pfizer | 0.5439 | 0.0213 | 0.4616 | 0.9999 | 0.0046 | 0.9982 |
| | monkey virus | | | | | | |
| 360107 | Campylobacter | 0.9713 | 0.0359 | 0.2413 | 0.9999 | 0.0084 | 0.9982 |
| | hominis ATCC | | | | | | |
| | BAA-381 | | | | | | |
| 838 | Prevotella | 0.7246 | 0.0482 | 0.2844 | 0.9999 | 0.0037 | 0.9982 |

A multivariate linear regression model utilizing the 11 microbial RNAs was also able to accurately predict collection time in all 3 sample sets, with Multiple R values ranging from 0.770 0.927 and Adjusted R2 values ranging from 0.468-0.732 (Table 1). As with the miRNA model, a non-linear relationship between the time of collection and microbial RNA concentrations in sample set 3 reduced the overall accuracy of the microbial model across the full 24 hour time cycle compared to when the 4 a.m. samples were removed from analysis (Adjusted R2=0.468 vs 0.624, Table 1), which yielded results comparable to those seen in sample Sets 1 and 2. Likewise, inspection of the alpha (intercept) and beta (slope) coefficient terms across the independent sample set regressions again indicated a very high degree of internal consistency in these models with highly significant correlations present between all sets of model term comparisons (Table 1, lower).

3. Relationship between CircaMiRs and CircaMicrobes. Relationships between levels of the 11 CircaMiRs and the 11 microbes with oscillating transcriptional activity were assessed across all 120 samples from sample Sets 1, 2, and 3 using a Pearson's correlation analysis. With the exception of one CircaMiR (miR-200b-3p) and one CircaMicrobe (Macrococcus caseolyticus), the CircaMiRs and CircaMicrobes were generally segregated by hierarchical clustering of expression patterns (FIG. 4). However, 5/11 (45%) CircaMiRs and 4/11 (36%) CircaMicrobes demonstrated significant (|R|>0.40, FDR<0.0001) associations. Three of these relationships involved direct associations (miR-8089/Micrococcus caseolyticus, miR-200b-3p/Fusobacterium nucleatum subsp. nucleatum, and miR-200b-3p/Falconid herpesvirus 1). There were five inverse associations between CircaMiRs and CircaMicrobes (miR-221-3p/Falconid herpesvirus 1, miR-128-3p/Fusobacterium nucleatum subsp. nucleatum, miR-128-3p/Fusobacterium nucleatum subsp. vincentii, miR-345-5p/Fusobacterium nucleatum subsp. nucleatum, miR-345-5p/Falconid herpesvirus 1).

4. CircaMiR Target Genes. Functional analysis of the 11 CircaMiRs in DIANA miRPath revealed 1265 high confidence (p<0.05, Micro-T threshold≥0.95) mRNA targets with enrichment for 22 KEGG pathways (Table 3). Notably, 11/22 KEGG pathway targets were involved in cell signaling. Interestingly, circadian rhythm was not among the KEGG pathways targeted by the 11 CircaMiRs according to this analysis. However, of the 30 human mRNAs in the circadian rhythm KEGG pathway (hsa04710), four (13%; Csnk1e, Rora, BHLHE40, and Prkaa2) were targeted by the 11 CircaMiRs. To more closely examine the potential relationship of CircaMiRs and circadian function, we expanded the analysis to the initial 61 CircaMiRs and used IPA software (which included additional circadian mRNA targets). The results revealed a significant overlap in Circadian Rhythm Signaling targets (13/34 mRNAs, 38%, p=2.2e-38) based on moderate-to-high probability predicted interactions, or experimentally-observed interactions. A complete list of the 28 mRNA transcript isoforms encompassing the 13 mRNAs and their 37 CircaMiR interactors was obtained.

The MiRpath target mapping tool also failed to detect enrichment of KEGG pathways involved in immune function or bacterial regulation among the 11 CircaMiR targets (or the 5 CircaMiRs with microbial associations in FIG. 4). However, several of the CircaMiRs that mapped to circadian genes were found to target mRNAs that were clearly involved in immune function. Subsequent interrogation of the protein-protein interaction network for all 1127 unique mRNA targets of the 11 most robust CircaMiRs using STRING software, revealed 3794 edges (interactions) with a clustering coefficient of 0.32. This exceeds the number of protein-protein interactions expected by chance alone (p=1.0E-16), and implies inter-relatedness of CircaMiR targets. Among the expected protein targets, 471 were involved in regulation of metabolic process (GO:0019222; FDR=8.5E-23), 413 were involved in regulation of macromolecule metabolic process (GO:0060255; FDR=4.9E-22), and 425 were involved in regulation of cellular metabolic process (GO:0031323; FDR=8.9E-22

TABLE 3
Physiologic pathways over-represented
by mRNA targets of the 11 CircaMiRs

| KEGG pathway | p-value | # genes | # miRNAs |
|---|---|---|---|
| Rap1 signaling pathway | 7.7E−05 | 30 | 9 |
| Mucin type O-Glycan biosynthesis | 1.5E−03 | 4 | 4 |
| Ras signaling pathway | 2.1E−03 | 30 | 8 |
| Estrogen signaling pathway | 2.1E−03 | 14 | 7 |
| Lysine degradation | 2.5E−03 | 6 | 6 |
| ErbB signaling pathway | 3.3E−03 | 17 | 7 |
| PI3K-Akt signaling pathway | 3.8E−03 | 38 | 9 |
| Proteoglycans in cancer | 4.7E−03 | 23 | 7 |
| Neurotrophin signaling pathway | 4.9E−03 | 19 | 7 |
| Choline metabolism in cancer | 5.2E−03 | 15 | 8 |
| Renal cell carcinoma | 1.2E−02 | 12 | 6 |
| mTOR signaling pathway | 1.5E−02 | 11 | 6 |
| Prolactin signaling pathway | 1.5E−02 | 11 | 7 |
| MAPK signaling pathway | 1.5E−02 | 29 | 8 |
| FoxO signaling pathway | 2.0E−02 | 17 | 7 |
| Long-term potentiation | 2.5E−02 | 11 | 7 |
| Endocytosis | 2.5E−02 | 21 | 8 |
| Focal adhesion | 3.6E−02 | 23 | 8 |
| Oocyte meiosis | 3.6E−02 | 14 | 7 |
| Protein processing in endoplasmic | 4.6E−02 | 18 | 5 |
| reticulum | | | |
| Insulin signaling pathway | 4.6E−02 | 17 | 5 |
| Glutamatergic synapse | 5.0E−02 | 13 | 5 |

Transcript Overlaps. Of the 1265 mRNAs targeted by the 11 CircaMiRs with high confidence (micro-T-cds score≥0.950), 38 were reliably detected in saliva (counts≥10 in 10% of samples) with small RNA sequencing at 50 base pairs. Among these 38 mRNAs, the salivary levels of 8 (21%) were significantly associated (FDR<0.05) with their CircaMiR counter-parts (Table 4). Two mRNAs were positively associated with miR-130b-3p (ATXN1, FOSL2), three were positively associated with miR-142-5p (GRIN2B, MSL2, NAMPT), one was negatively associated with 181c-5p (WASL), and two were positively associated with miR-200b-3p (YOD1, YWHAG). The strongest relationship was observed between miR-142-5p and GRIN2B (R=0.53, FDR=8.71E-09, Target score=0.984), a member of the Circadian Rhythm Signaling pathway in IPA.

TABLE 4
Transcripts targeted by CircaMiRs with associated expression levels across time

| | | | | | | Micro-CDS |
|---|---|---|---|---|---|---|
| MicroRNA | Gene | R | T-stat | p-value | FDR | Target Score |
| miR-130b-3p | ATXN1 | 0.37395 | 4.3799 | 2.59E−05 | 0.000129 | 0.963 |
| miR-130b-3p | FOSL2 | 0.49302 | 6.1557 | 1.06E−08 | 1.23E−07 | 0.969 |
| miR-142-5p | GRIN2B | 0.53012 | 6.7914 | 4.76E−10 | 8.71E−09 | 0.984 |
| miR-142-5p | MSL2 | 0.42696 | 5.129 | 1.16E−06 | 6.49E−06 | 0.981 |
| miR-142-5p | NAMPT | 0.51006 | 6.4417 | 2.67E−09 | 3.5E−08 | 0.969 |
| miR-181c-5p | WASL | −0.2945 | −3.3476 | 0.001094 | 0.002056 | 0.966 |
| miR-200b-3p | YOD1 | 0.23098 | 2.5788 | 0.011142 | 0.031478 | 0.973 |
| miR-200b-3p | YWHAG | 0.29093 | 3.3032 | 0.001266 | 0.005204 | 0.985 |

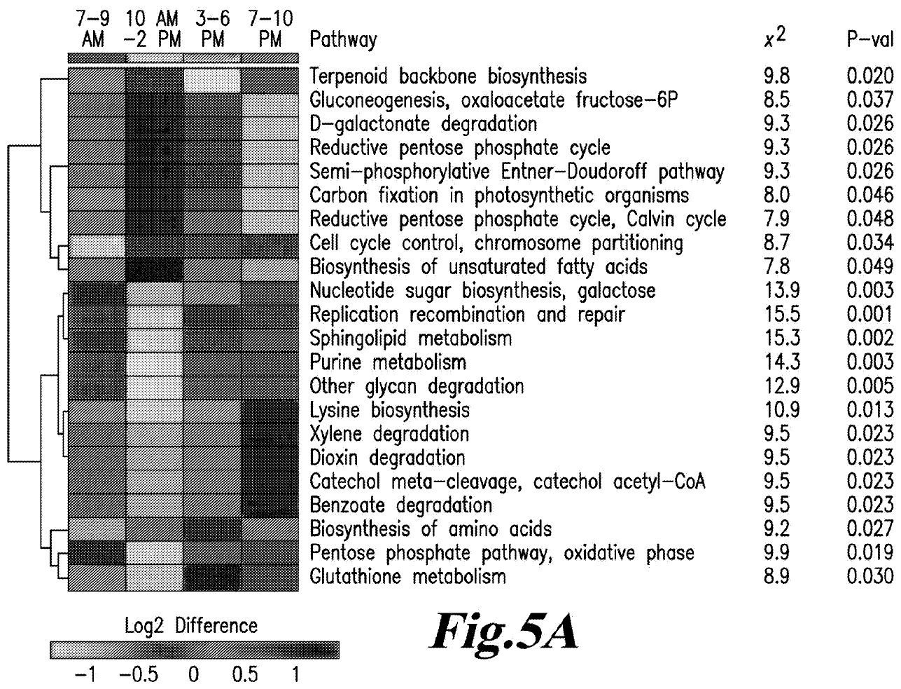

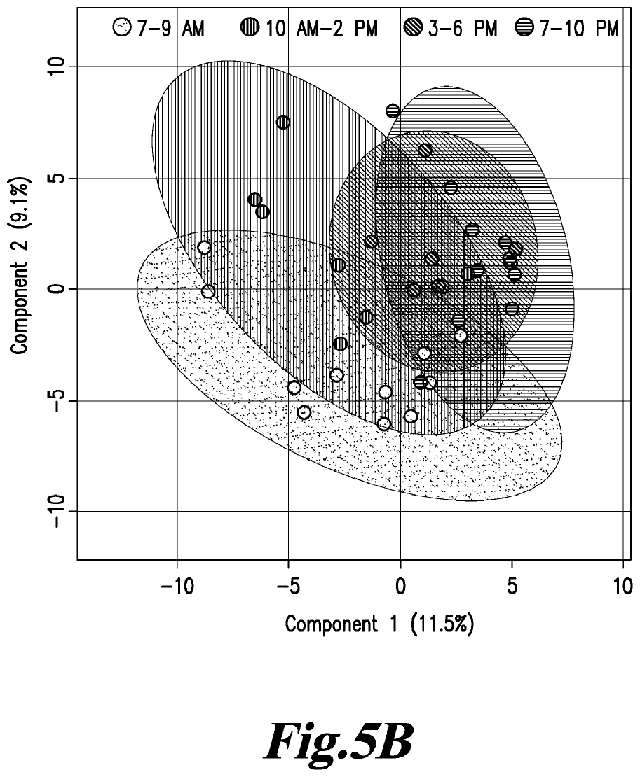

Metabolic targets of the oral microbiome. RNA expression patterns of oral microbes from the 9 participants in sample Sets 1, 2, and 3 were examined for evidence of diurnal variations in metabolic and functional clusters across four time periods: 7-9 a.m., 10 a.m.-2 PM, 3-6 PM, and 7-10 PM. Among the 202 functional clusters targeted by microbial RNAs, 22 pathways demonstrated nominal (p<0.05) differences in representation across the four time periods (FIG. 5A). Four of these functional pathways (nucleotide sugar biosynthesis, galactose; replication recombination and repair; sphingolipid metabolism; and purine metabolism) survived multiple testing corrections (FDR≤0.15). Among the 22 functional pathways with nominal changes, a cluster of seven pathways was up-regulated mid-day (10 a.m.-2 PM), while 10 pathways demonstrated diurnal peaks in the morning (7-9 AM) and evening (7-10 PM). Visualization of functional pathway expression differences in a partial least squared discriminant analysis resulted in partial separation of the four time periods, while accounting for 20.6% of the variance in COG/KEGG data in two dimensions (FIG. 5B).

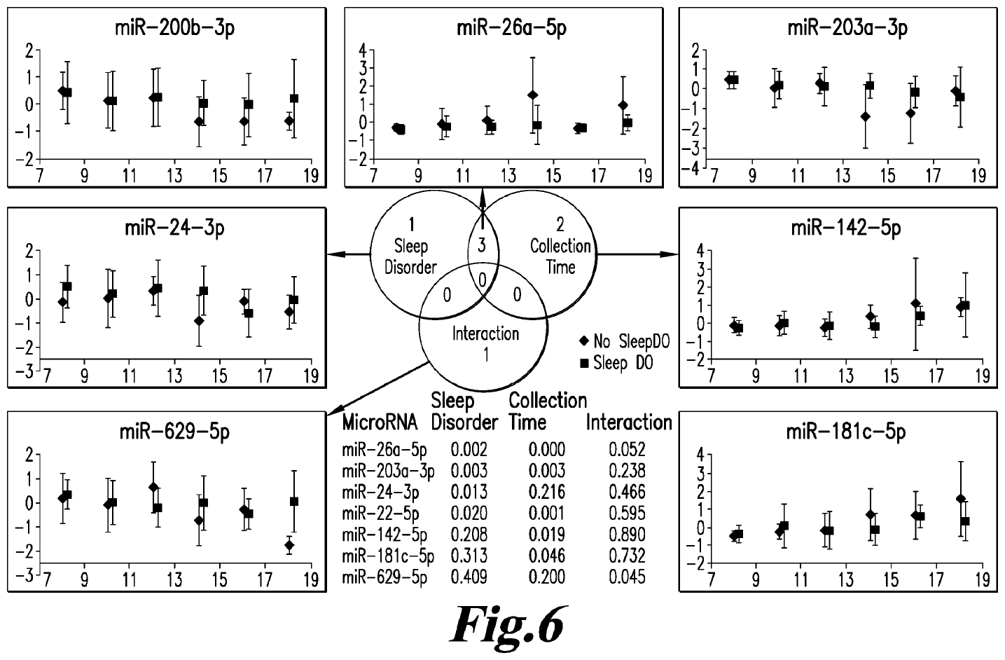

Measuring CircaMiR levels in children with disordered sleep. Differences in salivary miRNA expression between autistic children with (n=77) and without (n=63) disordered sleep was assessed with Mann Whitney U-test. Among the 61 CircaMiRs three demonstrated differences (FDR<0.05) between the two groups (miR-26a-5p, miR-24-3p, miR-203a-3p; Because this approach could not account for phase shifts in diurnal miRNA expression, salivary miRNA levels in the ASD cohort were also assessed with a 2-way ANOVA accounting for sleep disorder diagnosis and saliva collection time. This ANOVA analysis included the 11 robust CircaMiRs and the three miRNAs identified on Mann-Whitney testing. Among these 14 miRNAs, 4 demonstrated a significant interaction (p<0.05) with sleep disorder diagnosis (miR-24-3p, miR-200b-3p, miR-203a-3p, miR-26a-5p), 5 demonstrated a significant interaction with collection time (miR-142-5p, miR-181c-5p, miR-200b-3p, miR-203a-3p, miR-26a-5p), and 3 were affected by both factors (FIG. 6). We also detected a significant interaction between collection time and sleep disorder diagnosis for one CircaMiR (miR-629-5p).

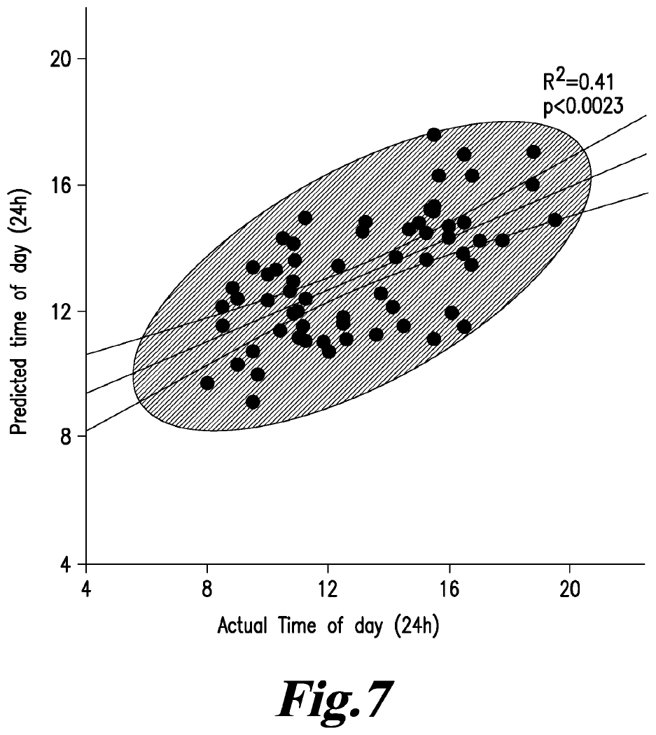

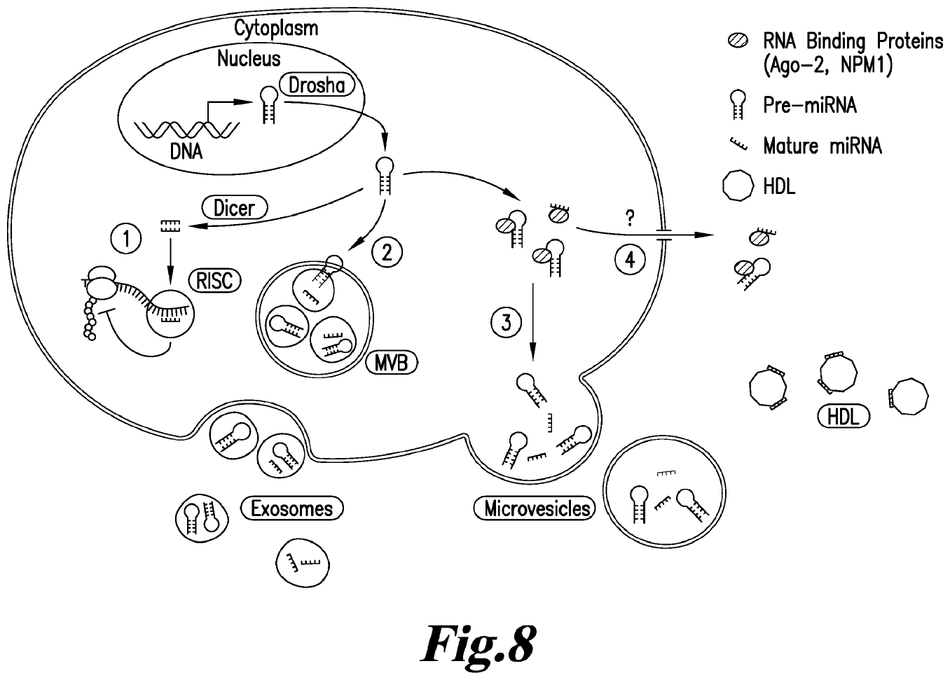

Based on the ability of the 11 CircaMiRs to predict time of collection in 11 typically developing, healthy children (and adults) in sample sets 1, 2 and 3, we also used a multivariate regression model examining their ability to predict time of collection in the 63 children with ASD and a normal sleep pattern, and the 77 children with ASD and comorbid disordered sleep. As we had seen in sample sets 1, 2 and 3, these 11 CircaMiRs yielded a significant regression (R²=0.41, F1,11=3.19, p<0.0023) that accurately predicted the time of collection with a mean absolute error of 15.25% (FIG. 7). Inspection of the multivariate regression coefficients and T scores indicated that no individual miRNA was significant in the presence of the others, although three showed strong trends (miR-629-5p, miR-22-5p, and miR-128-3p) (Table 5). In contrast to the significant regression findings for the ASD children without sleep disorder (n=63), the regression results for the ASD children with sleep disorders (n=77) using the 11 CircaMiRs did not yield a significant result (R²=0.20, F1,11=1.46, p>0.167).

TABLE 5
Prediction of collection time in ASD children
with normal sleep patterns (n = 63)

| Variable | T-stat | P | Power |
|---|---|---|---|
| hsa-miR-128-3p | 1.824 | 0.074 | 0.432 |
| hsa-miR-130b-3p | 0.055 | 0.956 | 0.050 |
| hsa-miR-140-3p | −0.250 | 0.803 | 0.057 |
| hsa-miR-142-5p | 0.794 | 0.431 | 0.122 |
| hsa-miR-181c-5p | 0.489 | 0.627 | 0.077 |
| hsa-miR-200b-3p | −0.892 | 0.377 | 0.141 |
| hsa-miR-22-5p | −1.860 | 0.069 | 0.446 |
| hsa-miR-221-3p | −0.899 | 0.373 | 0.143 |
| hsa-miR-345-5p | −0.110 | 0.913 | 0.051 |
| hsa-miR-629-5p | −1.961 | 0.055 | 0.486 |
| hsa-miR-8089 | 0.983 | 0.331 | 0.161 |

Relationships of CircaMiRs and CircaMicrobes with daily activities. Pearson's correlation analysis was used to explore relationships between oscillating salivary RNAs and three daily routines (sleep, tooth brushing, and eating) in sample set 3. Levels of 3 CircaMiRs and 5 CircaMicrobes were significantly (FDR<0.05) associated with time since last sleep (measured in hours; Table 6). Levels of all five CircaMicrobes were inversely associated with time since sleep, while 2/3 CircaMiRs were positively correlated with time since last sleep. There were 4 CiraMiRs and 5 CircaMicrobes associated with time since last tooth brushing. Levels of all five CircaMicrobes were inversely associated with time since last tooth brushing, while 3/4 CircaMiRs were positively associated with time since last tooth brushing. MiR-200b-3p was the single CircaMiR inversely associated with both sleep and tooth brushing. Notably, expression patterns of miR-200b-3p were also hierarchically clustered with CircaMicrobe expression (FIG. 4). Two Prevotella and two Fusibacterium CircaMicrobes were associated with both sleep and tooth brushing. There was only one CircaMicrobe positively associated with time since last meal (and 0 CircaMiRs).

TABLE 6
Relationship between oscillating salivary RNA levels and timing of daily activities.

| | Hours awake since | | | Hours since last | | | Hours since last | | |
|---|---|---|---|---|---|---|---|---|---|
| | last sleep | | | toothbrush | | | meal | | |
| RNA Component | R | FDR | T-stat | R | FDR | T-stat | R | FDR | T-stat |
| Microbial RNA | | | | | | | | | |
| Flaconid herpesvirus 1 | −0.48 | 0.00 | −3.75 | −0.18 | 0.36 | −1.23 | 0.09 | 0.75 | 0.63 |
| Prevotella | −0.39 | 0.03 | −2.88 | −0.50 | 0.00 | −3.92 | 0.23 | 0.33 | 1.58 |
| melaninogenica ATCC | | | | | | | | | |
| 25845 | | | | | | | | | |
| Haemophilus | 0.18 | 0.40 | 1.21 | 0.24 | 0.19 | 1.69 | 0.01 | 0.97 | 0.07 |
| parainfluenzae T3T1 | | | | | | | | | |
| Veillonella parvula | −0.36 | 0.05 | −2.60 | −0.15 | 0.44 | −1.06 | 0.36 | 0.11 | 2.59 |
| DSM 2008 | | | | | | | | | |
| Macrococcus | 0.28 | 0.14 | 1.96 | 0.30 | 0.09 | 2.12 | 0.02 | 0.94 | 0.16 |
| caseolyticus JCSC5402 | | | | | | | | | |
| Fusobacterium | −0.57 | 0.00 | −4.76 | −0.61 | 0.00 | −5.24 | 0.24 | 0.30 | 1.69 |
| nucleatum subsp. | | | | | | | | | |
| nucleatum 25586 | | | | | | | | | |
| Haemophilus | 0.17 | 0.41 | 1.19 | 0.32 | 0.07 | 2.30 | −0.04 | 0.90 | −0.27 |
| Fusobacterium | −0.46 | 0.01 | −3.51 | −0.50 | 0.00 | −3.88 | 0.29 | 0.21 | 2.07 |
| nucleatum subsp. | | | | | | | | | |
| vincentii | | | | | | | | | |
| Mason-Pfizer monkey | −0.35 | 0.05 | −2.56 | −0.18 | 0.36 | −1.24 | 0.38 | 0.09 | 2.77 |
| virus | | | | | | | | | |
| Campylobacter hominis | −0.24 | 0.22 | −1.67 | −0.47 | 0.00 | −3.58 | 0.44 | 0.04 | 3.34 |
| ATCC | | | | | | | | | |
| Prevotella | −0.47 | 0.01 | −3.58 | −0.60 | 0.00 | −5.03 | 0.30 | 0.19 | 2.13 |
| Human microRNA | | | | | | | | | |
| miR-142-5p | 0.37 | 0.04 | 2.71 | 0.42 | 0.01 | 3.13 | −0.16 | 0.53 | −1.09 |
| miR-130b-3p | 0.12 | 0.60 | 0.81 | −0.04 | 0.87 | −0.26 | −0.22 | 0.34 | −1.55 |
| miR-629-5p | −0.04 | 0.88 | −0.26 | −0.29 | 0.11 | −2.04 | 0.05 | 0.88 | 0.32 |
| miR-140-3p | 0.35 | 0.05 | 2.55 | 0.23 | 0.23 | 1.57 | −0.35 | 0.12 | −2.49 |
| miR-128-3p | 0.35 | 0.05 | 2.54 | 0.30 | 0.09 | 2.11 | −0.35 | 0.12 | −2.50 |
| miR-181c-5p | 0.25 | 0.19 | 1.76 | 0.16 | 0.42 | 1.10 | −0.37 | 0.09 | −2.70 |
| miR-345-5p | 0.70 | 0.00 | 6.70 | 0.58 | 0.00 | 4.83 | −0.38 | 0.09 | −2.78 |
| miR-22-5p | 0.35 | 0.05 | 2.50 | 0.35 | 0.04 | 2.52 | 0.01 | 0.98 | 0.04 |
| miR-8089 | −0.01 | 0.96 | −0.09 | 0.08 | 0.70 | 0.55 | 0.11 | 0.68 | 0.77 |
| miR-221-3p | 0.21 | 0.30 | 1.45 | 0.07 | 0.75 | 0.46 | 0.03 | 0.92 | 0.23 |
| miR-200b-5p | −0.44 | 0.01 | −3.31 | −0.43 | 0.01 | −3.26 | 0.21 | 0.39 | 1.44 |

Discussion. In the present study, 61 total human miRNAs (CircaMiRs) and 11 total microbes (CircaMicrobes) displayed consistent diurnal oscillations in saliva samples obtained from 9 different children and adults collected across multiple days and times. From these, 11 miRNAs and 11 microbes were capable of accurately and reliably predicting time of sample collection. Diurnal levels of five CircaMiRs and four CircaMicrobes were strongly associated with one another. Functional analyses of the circadian RNA components displayed enrichment for numerous signaling mechanisms, particularly metabolic pathways. However, CircaMiR and CircaMicrobe levels were more strongly associated with sleep routines than with eating routines. This may explain, partly, why levels of four CircaMiRs were “altered” in autistic children with disordered sleep, relative to autistic peers without a sleep disorder and why a portion of these CircaMiRs target circadian mRNAs.

Six of the oscillating miRNAs identified in this study (miR-15b-3p, miR-24-3p, miR-106b-3p, miR-140-3p, miR-150-5p, miR-203a-3p) were among the 26 plasma miRNAs previously found to have diurnal variations in peripheral blood samples from healthy individuals [24]. These overlapping miRNAs from two distinct biofluids may represent either primary regulatory elements or primary readouts of circadian rhythmicity. This premise is supported by the fact that levels of both miR-24-3p and miR-203a-3p are disrupted in the cohort of autistic children with disordered sleep patterns. In addition, we found suggestive evidence that miR-203a-3p was associated with sleep initiation difficulties (R=0.20; p=0.034). Another CircaMiR, miR-142-5p, targets the clock gene RORA. Notably, miR-142-5p also displays correlated diurnal expression with its mRNA targets NAMPT (whose gene product modulates circadian clock function by releasing the CLOCK/ARNTL/BMAL heterodimer [29]) and GRIN2B (whose gene product encodes the NR2B subunit of the NDMA receptor essential to MAPK signaling in the suprachiasmatic nucleus and CaMK II signaling in the hippocampus [30]). Notably, a well-described developmental switch from NR2A to NR2B subunit expression is considered a hallmark of synaptic maturation that promotes memory formation, and elevation in miR-142-5p (which would suppress NR2B expression) is associated with amyloid beta pathology in postmortem brain samples of subjects with Alzheimer's disease (AD) [31]. The importance of this finding is highlighted by the fact that AD is associated with significant circadian pathology (e.g. “sundowning”) and that miR-142-5p restores normal synapse formation and maturation (as measured by PSD95 expression) in differentiated neural cultures [32]. Such a mechanism might even contribute to the recently described circadian oscillation in synaptic spine number that has been described across different species, especially dendritic spines on inhibitory neurons in multiple brain regions [33-35].

Circadian miRNAs found in both plasma and saliva may also direct diurnal physiologic processes common to both peripheral biofluids. Indeed, mapping of KEGG pathway targets for the six overlapping miRNAs reveals enrichment for broad signaling mechanisms such as Wnt Signaling, Rap1 signaling, and Endocrine factor-regulated calcium reabsorption. This is consistent with functional analysis of the 11 CircaMiRs, which also display enrichment for Rap1 and other broad signaling processes (Ras, ErbB, PI3K-Akt, mTOR, MAPK). Salivary CircaMiRs also demonstrate target enrichment for endocrine factors (estrogen and prolactin signaling), which regulate peripheral physiologic processes in a circadian manner [36].

Unlike oscillating miRNAs in plasma, the CircaMiRs and CircaMicrobes in saliva appear uniquely geared toward metabolic functions. CircaMiR targets display enrichment for lysine degradation, choline metabolism, and insulin signaling (Table 3). The protein products of these mRNA targets also exhibit enhanced biologic interaction in metabolism at the cellular and macromolecule levels Specifically, interactions between miR-130b-3p/ATXN2, and miR-142-5p/NAMPT (Table 4) may play important roles in regulation of host metabolism, given that loss of function mutations in both ATXN2 and NAMPT are associated with obesity and diabetes mellitus [37, 38].

Oscillating RNA expression within the oral microbiome also shows relationships with diurnal metabolism. Microbial RNAs appear to target KEGG and COG pathways in a diurnal manner, by up-regulating RNAs involved in terpenoid biosynthesis, gluconeogenesis, pentose phosphate pathways, and carbon fixation during the morning and afternoon time periods. In comparison, pathways related to cell replication, nucleotide biosynthesis, and purine metabolism demonstrate both morning and evening peaks. Thus, as a whole, the oral microbiome may have evolved energy utilization patterns that capitalize on the timing of host meals to extract biosynthetic materials and allow for night time replication. Interestingly, however, levels of the 11 CircaMicrobes do not appear to correlate with time since last meal. Thus, these 11 individual entities may serve a more commensal function whose metabolic activities aid host circadian rhythms. Indirect evidence for this may be found in the circadian rhythm of terpenoid biosynthesis (FIG. 5A), a diverse class of hydrocarbons present in plant-based cannabinoids, or anti-inflammatory circuminoids that play an essential role in steroid production [39]. Given the well-established rhythmicity of steroid production, this is one mechanism by which the microbiome may contribute to host circadian biology [40].

Further evidence for a synergistic relationship between CircaMicrobes and human hosts is found in the strong associations among CircaMiR and CircaMicrobe expression (FIG. 4). It is somewhat surprising that CircaMiRs have few immune, or antimicrobial targets. However, this may be because the circadian components of the oral microbiome serve a commensal function. The majority of CircaMicrobes are not known to play pathogenic roles in human hosts. Of the 11 CircaMicrobes, only three are distinct human pathogens (Haemophilus parainfluenza T3T1, Haemophilus, and Campylobacter hominis ATTC BAA-381) and none of these three are associated with CircaMiR levels. Instead CircaMiRs may interact with the oral microbiome to coordinate metabolic patterns, or production of essential amino acids. Perhaps metabolic activity by the oral microbiome leads to changes in host miRNAs that regulate downstream physiologic pathways.

To our knowledge this is only the second study to report consistent circadian rhythmicity of peripheral miRNA expression in humans, and the first to do so in saliva. The importance of this finding is underscored by the vast number of publications seeking to use peripheral miRNA expression as a biological marker of human disease [41], a venture that could be greatly confounded by failure to control for time of collection. For example, among seven studies describing peripheral miRNA expression (saliva, serum, lymphoblasts [42]) in patients with ASD, none have reported, or controlled for time of RNA collection. These studies have reported a combined 139 ASD-related miRNAs. Notably, 10 of these (7%) overlap with the 61 CircaMiRs identified herein, which could represent confounded results. Future studies may be able to utilize CircaMiR levels to control for circadian variation in miRNA expression or accurately identify time of collection among biofluid samples.

The current study also adds to the growing body of literature that suggests miRNAs may serve as a communication mechanism between the gut microbiome and human hosts [43]. Specifically, these results show how miRNA-microbiome cross-talk may occur in a circadian manner. Given the diurnal rhythmicity of human metabolism, this finding has implications in human health and disease. For instance, daily fluctuations in host-microbiome interaction may inform risk for obesity, or insulin resistance (an enriched KEGG target of the 11 CircaMiRs). Alternatively, disruptions in miRNA-microbiome networks may unsettle the gut-brain-axis, a concept implicated in diseases such as Parkinson's [44] and ASD [45] (both of which are associated with disordered sleep).

There are several notable limitations to this study that must be considered in interpreting these findings. Detailed daily activity logs were available only for the participants in sample Set 3. The remaining participants reported no medical co-morbidities (including disordered sleep), though timing of sleep initiation and cessation were not recorded. Such information, when recorded alongside physiologic measurements such as sleep architecture, or melatonin flux could be extremely informative when interpreting RNA results in future studies. Nonetheless, the RNA expression patterns from participants in sample sets 1 and 2 were sufficient to accurately predict collection time in a third independent sample set with documented sleep wake cycles.

Notably, predictive performance in sample set 3 was somewhat impaired for the subset of samples obtained at 4 AM. This may have resulted because the sinusoidal model created from samples collected between 8 a.m. and 8 p.m. could not fully account for the overnight rhythmicity that occurs in a sleep state. There may also be microbial variability introduced by differences in participant breathing patterns (e.g. open-mouthed versus nasal breathing) or fasting during sleep. Certainly, a more controlled study which tightly dictated wake time, sleep initiation time, diet, dental hygiene, and other factors could account for time of collection with greater precision. However, the current results demonstrate that even in the face of typical variability among daily routines, these 11 miRNAs and 11 microbial RNAs are remarkably accurate predictors of time of saliva collection in four different cohorts of human subjects.

The accuracy of these results may even be underestimated given the broad age range (3-55) of participants in sample Sets 1, 2 and 3. The CircaMiR and CircaMicrobe candidates were generated from 2 cohorts of children and validated in a cohort of teens and adults. This is despite the fact that teens are known to have altered circadian rhythm compared with pre-teen peers and adults[46]. Circadian RNAs from sample sets 1-3 also demonstrated significant relationships with collection time in a large cohort of children with ASD. Thus, the age and developmental diversity of these sample sets may be viewed as a confounding variable, but it likely enhances the veracity of these results.

Finally, it should be noted that the RNAseq approach used to identify oral microbes and estimate transcriptional activity of individual taxons differs from the typical 16S approach used to measure microbial abundance. Thus, these results should not be interpreted as diurnal fluctuations in the quantity of the oral microbiome, but rather as circadian variation in salivary microbial activity. RNAseq and 16S measures are complementary (though not equivocal) and could potentially add to the interpretive value of this approach in future studies. Animal models may be used to explore the cellular origins of salivary CircaMiRs and investigate the mechanisms regulating CircaMiR production, transport, and degradation. Manipulating the gut microbiome in this setting may also provide insights into microbial-miRNA communication.

Parallel circadian oscillation in host and microbial RNA represents an important consideration for studies analyzing epi-transcriptomic or metagenomic mechanisms in human health and disease. Circadian rhythm disturbances are a common problem in disorders of the central nervous system (e.g. Parkinson's, Alzheimer's, autism, depression, concussion [47]) Hence, studies of peripheral miRNA expression in these conditions might consider how diurnal miRNA expression patterns are shifted, rather than simply focusing on average miRNA levels at a single collection point in comparison with a control cohort. Monitoring levels of these factors in biofluids like saliva could have diagnostic potential in diseases with altered circadian rhythm and may one day provide a basis for targeted miRNA therapy of circadian disruptions.

As shown herein, the inventors screened expression of salivary microRNAs and microbial mRNAs in healthy children and adults across multiple time points and days using next generation sequencing.

Sets of RNAs were identified that oscillate in circadian fashion and can be used to predict collection time with high precision and accuracy.

Subsets of these circadian miRNAs (CircaMiRs) were correlated with microbial RNAs and targeted human genes that regulate circadian function and metabolism.

Changes in functional metabolic microbial profiles across time were also identified. An independent sample of children with sleep disorders was found to demonstrate less robust predictability than peers without sleep disorders.

Collectively, the data show that regular daily oscillations in salivary miRNA and microbial RNA either direct or reflect changes in the upper GI-microbiome and can impact signaling processes related to human health and disease.

Statistical Analysis

A two-way analysis of variance (ANOVA) was performed in the Collection 1 and 2 sample sets to identify miRNAs and microbes that varied significantly according to collection time but not the day of collection (which could have been strongly affected by daily variation in routines). A subset of these miRNAs and microbes were then used in a third sample set to assess the accuracy of prediction for the time of collection using multivariate linear regression. miRNAs that showed the strongest circadian oscillations were termed circaMiRs and examined for being predicted regulators of a total of 139 annotated circadian genes using Ingenuity Pathway Analysis (IPA) software. circaMiRs targeting circadian genes were then examined for evidence of association with the strongest circadian-oscillating microbes using Pearson correlation analysis. The functions of the genes targeted by circaMiRs were examined for their specific biological functions using IPA and miRpath software.

Results

Preliminary results show that a difference in statistics (e.g., variance, total variance, or average variance) related to epigenetic data (e.g., miRNA and/or microbiome) and/or a difference in level of expression (e.g., read count, fluorescence, etc.) of epigenetic data may be used to distinguish between healthy subjects (children and/or adults) and subjects suffering from a particular disease, disorder, or condition. The particular diseases or disorders distinguishable based on the systems and methods described herein may be, without limitation, autism spectrum disorder (ASD), sleep disorders, and/or traumatic brain injury. In some embodiments, certain subjects (e.g., ASD patients) may have a lower average variance relative to normal, healthy subjects. In other embodiments, certain subjects may have a higher average variance relative to normal, healthy subjects.

A total of 38 miRNAs (Group B) in a 24 sample data set showed a highly-significant effect of collection time (FDR<0.001) and no effect of day of collection.

A total of 41 mi miRNAs in a 48 sample data set showed a highly-significant effect of collection time (FDR<0.001) and no effect of day of collection.

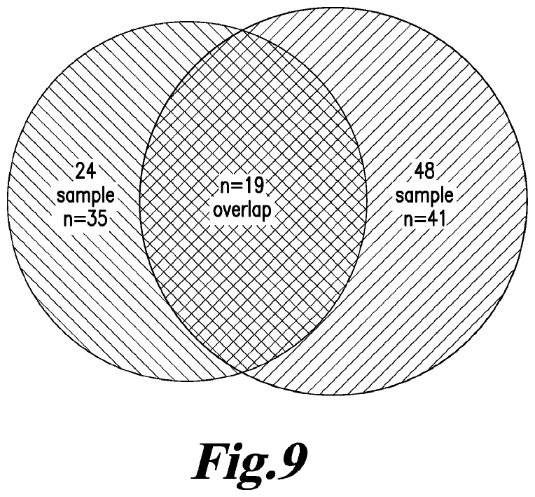

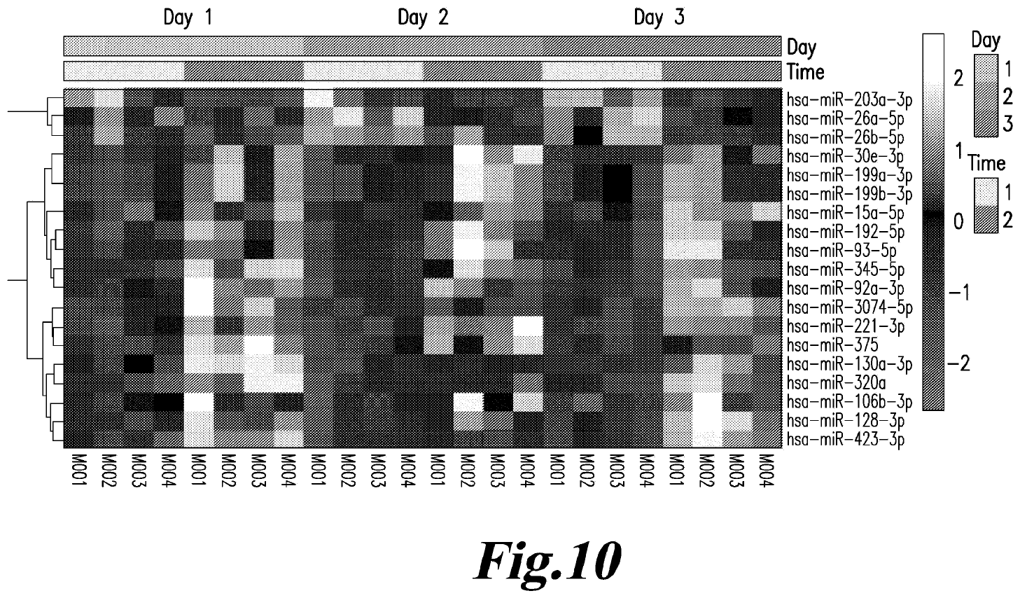

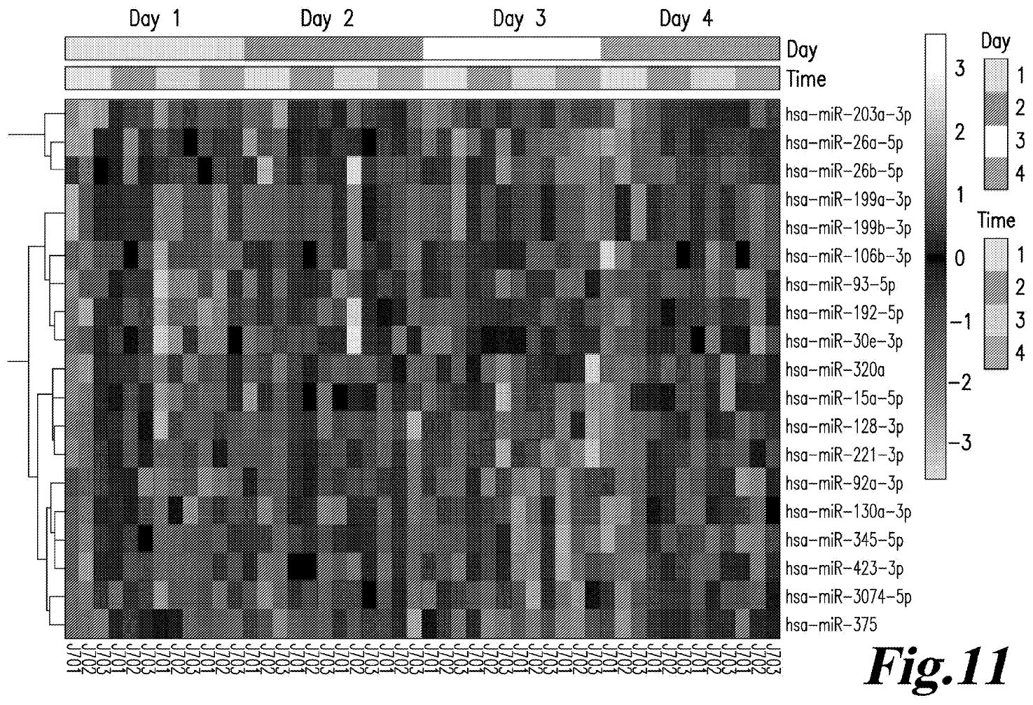

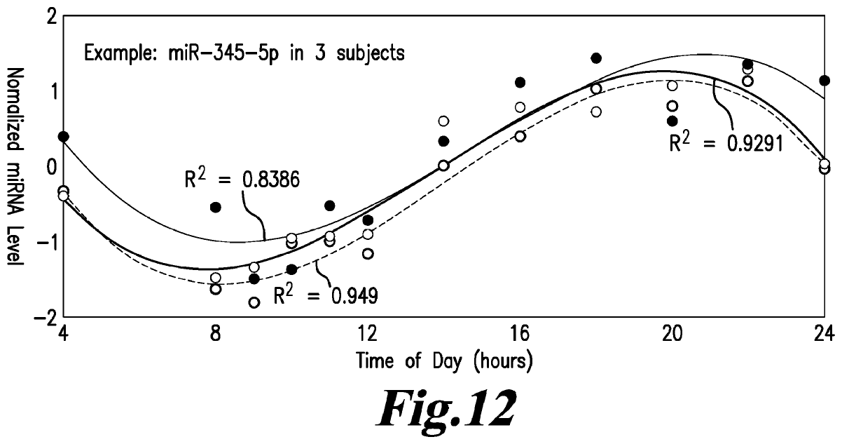

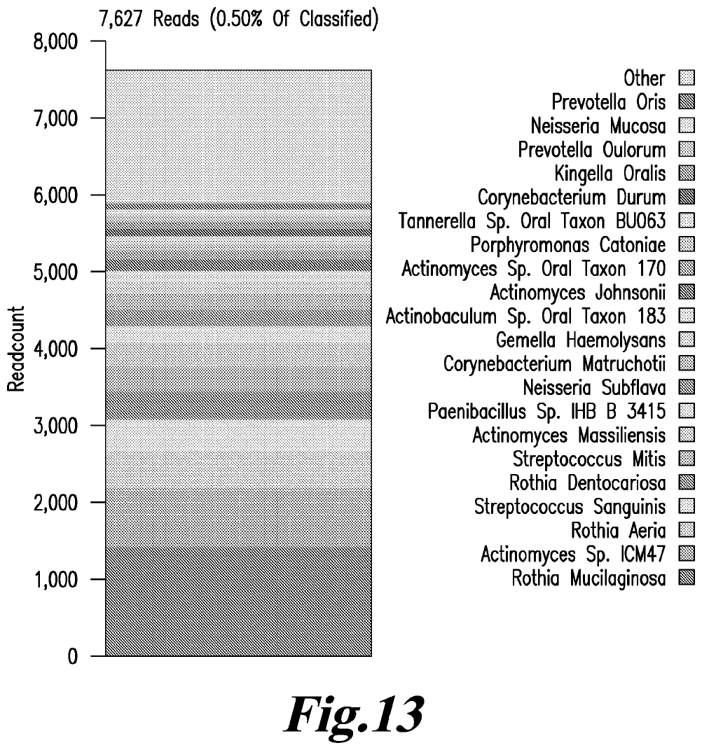

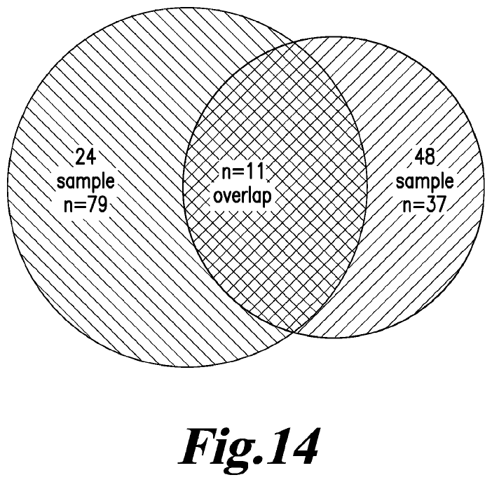

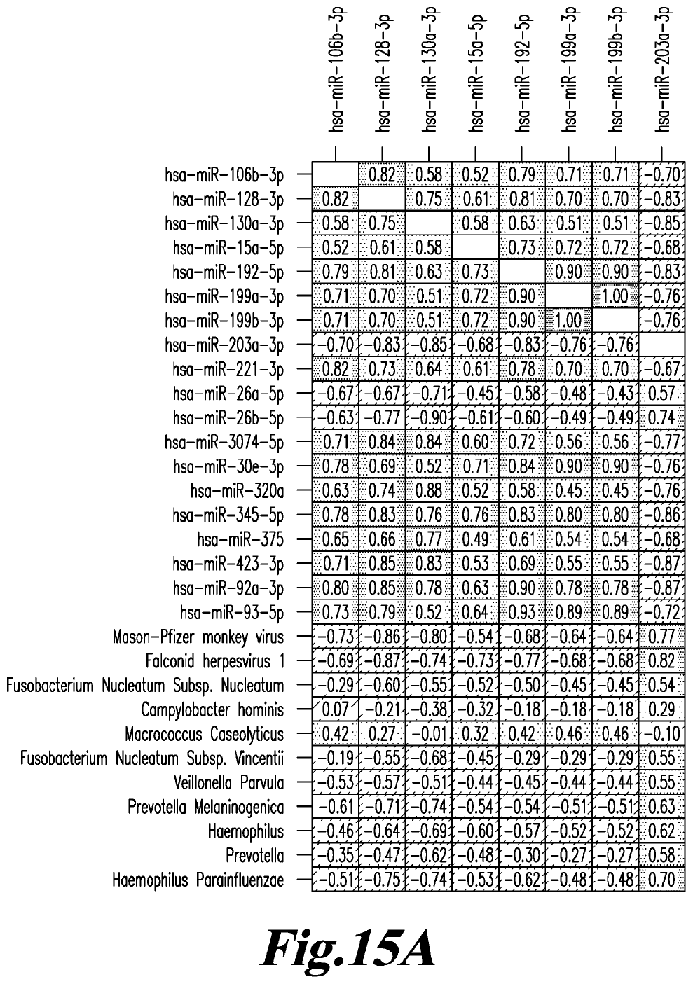

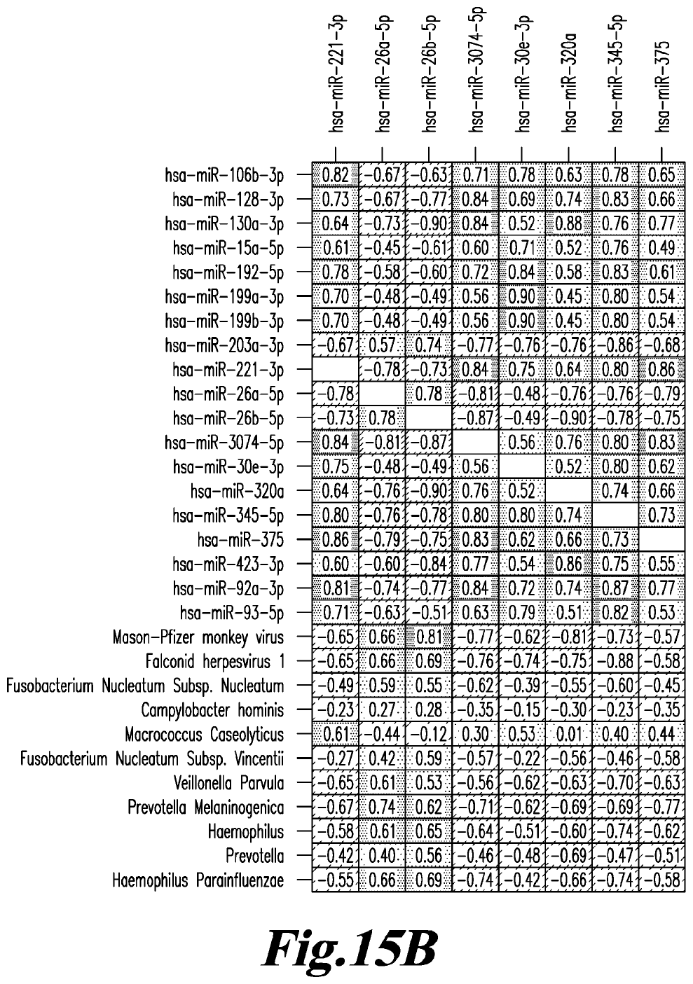

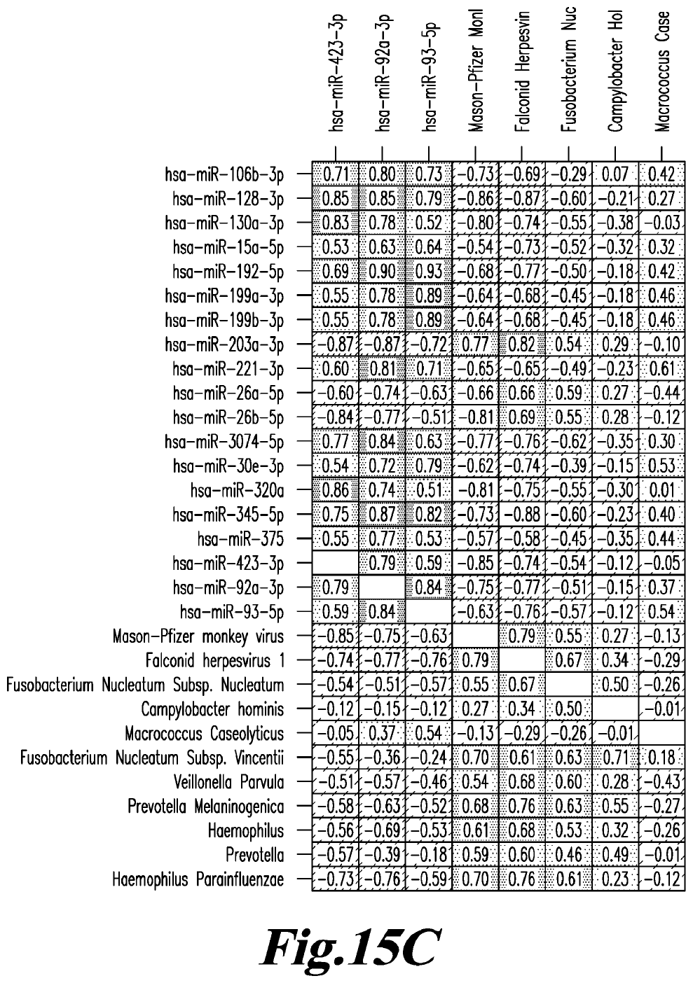

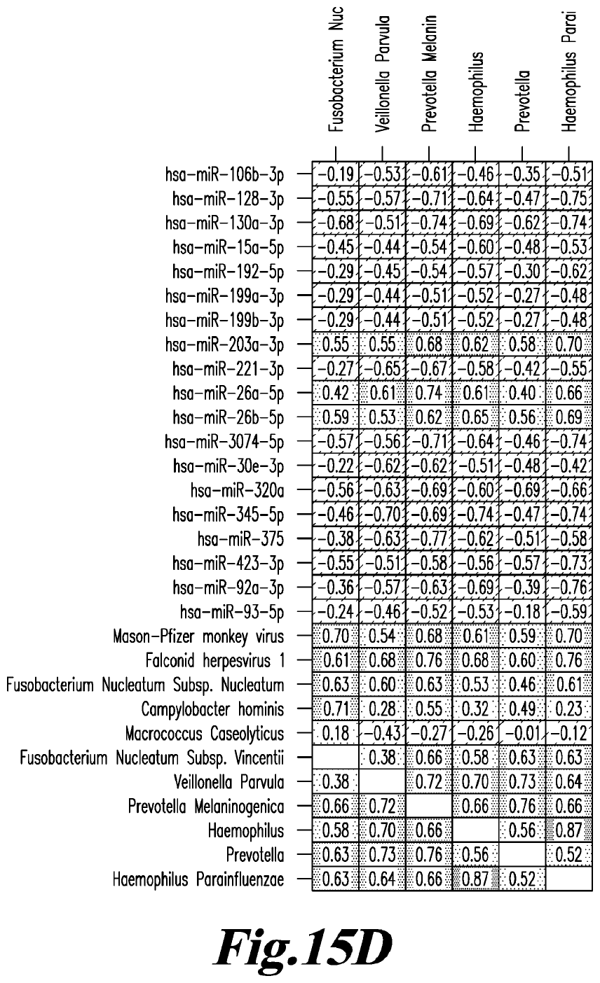

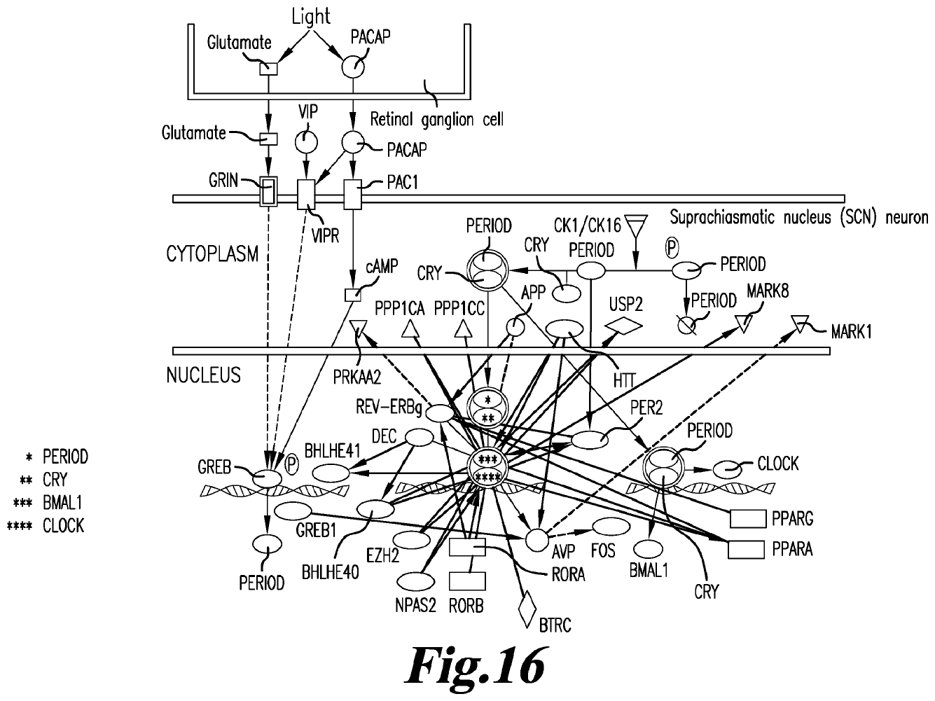

It was found that 19 miRNAs were commonly changed in both sets (see Group A in Table 2A). These were examined for the ability to predict collection time in a third data set as shown in FIG. 9).

CircaMiR Time Prediction

TABLE 1-2
Accuracy of 19 circaMiRs to predict collection time

| Table 1-2 | Multiple R | P value | Margin of Error |
|---|---|---|---|
| Collection 1 | 0.990 | 0.003929 | 12.9% |
| Collection 2 | 0.878 | 0.00031 | 18.1% |
| Collection 3 | 0.875 | 0.000040 | 26.0% |
| (no 4 a.m.) | 0.938 | 2.2e−¹⁰ | 15.7% |

Microbe Findings

TABLE 2-2
List of 11 microbes most related to collection time

| | | Sample Collection 1 | | | Sample Collection 2 | | |
|---|---|---|---|---|---|---|---|
| Taxon ID | Taxon name | Day | Time | Interaction | Day | Time | Interaction |
| 1510155 | Falconid herpesvirus 1 | 0.7246 | 0.0003 | 0.1104 | 0.9999 | 0.0009 | 0.9982 |
| 553174 | Prevotella melaninogenica ATCC | 0.8213 | 0.0011 | 0.1693 | 0.9999 | 0.0359 | 0.9982 |
| | 25845 | | | | | | |
| 862965 | Haemophilus parainfluenzae T3T1 | 0.2276 | 0.0061 | 0.2426 | 0.9999 | 0.0045 | 0.9982 |
| 479436 | Veillonella parvula DSM 2008 | 0.7246 | 0.0076 | 0.1069 | 0.9999 | 0.0001 | 0.9982 |
| 458233 | Macrococcus caseolyticus | 0.0830 | 0.0338 | 0.1302 | 0.9999 | 0.0381 | 0.9982 |
| | JCSC5402 | | | | | | |
| 190304 | Fusobacterium nucleatum subsp. | 0.9782 | 0.0127 | 0.1069 | 0.9999 | 0.0350 | 0.9982 |
| | nucleatum ATCC 25586 | | | | | | |
| 724 | Haemophilus | 0.5928 | 0.0127 | 0.2426 | 0.9999 | 0.0139 | 0.9982 |
| 469604 | Fusobacterium nucleatum subsp. | 0.7246 | 0.0209 | 0.1069 | 0.9999 | 0.0187 | 0.9982 |
| | vincentii 3136A2 | | | | | | |
| 11855 | Mason-Pfizer monkey virus | 0.5439 | 0.0213 | 0.4616 | 0.9999 | 0.0046 | 0.9982 |
| 360107 | Campylobacter hominis ATCC BAA- | 0.9713 | 0.0359 | 0.2413 | 0.9999 | 0.0084 | 0.9982 |
| | 381 | | | | | | |
| 838 | Prevotella | 0.7246 | 0.0482 | 0.2844 | 0.9999 | 0.0037 | 0.9982 |

TABLE 3-2
Accuracy of 11 microbes to predict collection time

| | Multiple R | P value | Margin of Error |
|---|---|---|---|
| Collection 1 | 0.927 | 0.00139 | 24.5% |
| Collection 2 | 0.858 | 1.73e⁻⁷ | 19.38% |
| Collection 3 | 0.709 | 0.003175 | 33.99% |
| (no 4 am) | 0.865 | 8.03e⁻⁷ | 20.7% |

Other Functions of circaMiRs

TABLE 4-2
Biological pathways containing genes targeted by circaMiRs

| Kyoto Encyclopedia of Genes and | | | |
|---|---|---|---|
| Genomes [KEGG] Pathways | p-value | # genes | # miRNAs |
| Fatty acid biosynthesis | 4.6e−11 | 5 | 6 |
| Proteoglycans in cancer | 3.1e−08 | 94 | 17 |
| Prion diseases | 4.8e−07 | 10 | 9 |
| Hippo signaling pathway | 2.0e−06 | 71 | 17 |
| FoxO signaling pathway | 8.0e−06 | 70 | 16 |
| Signaling pathways regulating | 8.0e−06 | 68 | 17 |
| pluripotency of stem cells | | | |
| Renal cell carcinoma | 1.1e−05 | 39 | 17 |
| Glutamatergic synapse | 7.9e−05 | 52 | 17 |
| Prostate cancer | 7.9e−05 | 47 | 17 |
| Pathways in cancer | 8.0e−05 | 159 | 17 |
| Glioma | 8.7e−05 | 33 | 15 |
| Adrenergic signaling in cardiomyocytes | 8.7e−05 | 61 | 17 |
| Estrogen signaling pathway | 0.00013 | 46 | 16 |
| Thyroid hormone signaling pathway | 0.00014 | 57 | 16 |
| Rap1 signaling pathway | 0.00016 | 91 | 17 |
| Regulation of actin cytoskeleton | 0.00027 | 94 | 17 |
| PI3K-Akt signaling pathway | 0.00044 | 136 | 17 |
| Focal adhesion | 0.00044 | 91 | 17 |
| mTOR signaling pathway | 0.00055 | 34 | 15 |

As shown above in the second method of analysis, portions of the saliva miRNA and microbiome levels show strong circadian patterns. This observation is surprising and has not been previously described. Moreover, these data show that there are highly significant correlations between several of the saliva miRNAs and microbes and that most saliva circaMiRs target at least one or more circadian genes in addition to genes involved in brain, metabolic and cancer function. Tables 2A and 2B above describe circaMiRs and microbiomes that may be used to distinguish healthy subjects from subjects having a condition, disorder or disease associated with an abnormal temporal rhythm using the methods described herein. Moreover, other miRNAs sharing the same seed sequences as any of the miRNAs in the above tables may be used to distinguish a healthy subject from a subject having a particular disease or disorder.

Terminology used herein is for the purpose of describing particular embodiments only and is not intended to be limiting of the invention.

The headings (such as “Background” and “Summary”) and sub-headings used herein are intended only for general organization of topics within the present invention, and are not intended to limit the disclosure of the present invention or any aspect thereof. In particular, subject matter disclosed in the “Background” may include novel technology and may not constitute a recitation of prior art. Subject matter disclosed in the “Summary” is not an exhaustive or complete disclosure of the entire scope of the technology or any embodiments thereof. Classification or discussion of a material within a section of this specification as having a particular utility is made for convenience, and no inference should be drawn that the material must necessarily or solely function in accordance with its classification herein when it is used in any given composition.

As used herein, the singular forms “a”, “an” and “the” are intended to include the plural forms as well, unless the context clearly indicates otherwise.

It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

As used herein, the term “and/or” includes any and all combinations of one or more of the associated listed items and may be abbreviated as “/”.

Links are disabled by deletion of http: or by insertion of a space or underlined space before www. In some instances, the text available via the link on the “last accessed” date may be incorporated by reference.

As used herein in the specification and claims, including as used in the examples and unless otherwise expressly specified, all numbers may be read as if prefaced by the word “substantially”, “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), +/−15% of the stated value (or range of values), +/−20% of the stated value (or range of values), etc. Any numerical range recited herein is intended to include all sub-ranges subsumed therein.

Disclosure of values and ranges of values for specific parameters (such as temperatures, molecular weights, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if parameter X is exemplified herein to have values in the range of 1-10 it also describes subranges for Parameter X including 1-9, 1-8, 1-7, 2-9, 2-8, 2-7, 3-9, 3-8, 3-7, 2-8, 3-7, 4-6, or 7-10, 8-10 or 9-10 as mere examples. A range encompasses its endpoints as well as values inside of an endpoint, for example, the range 0-5 includes 0, >0, 1, 2, 3, 4, <5 and 5.

As used herein, the words “preferred” and “preferably” refer to embodiments of the technology that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the technology.

As referred to herein, all compositional percentages are by weight of the total composition, unless otherwise specified. As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present invention that do not contain those elements or features.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

The description and specific examples, while indicating embodiments of the technology, are intended for purposes of illustration only and are not intended to limit the scope of the technology. Moreover, recitation of multiple embodiments having stated features is not intended to exclude other embodiments having additional features, or other embodiments incorporating different combinations of the stated features. Specific examples are provided for illustrative purposes of how to make and use the compositions and methods of this technology and, unless explicitly stated otherwise, are not intended to be a representation that given embodiments of this technology have, or have not, been made or tested.

### REFERENCES

1. Takeda, N. and K. Maemura, Circadian clock and the onset of cardiovascular events. Hypertension Research, 2016. 39: p. 383.
2. Reinke, H. and G. Asher, Circadian clock control of liver metabolic functions. Gastroenterology, 2016. 150(3): p. 574-80.
3. Videnovic, A. and P. C. Zee, Consequences of circadian disruption on neurologic health. Sleep Med Clin, 2015. 10(4): p. 469-80.
4. Buhr, E. D. and J. S. Takahashi, Molecular components of the mammalian circadian clock. Handb Exp Pharmacol, 2013(217): p. 3-27.
5. Stoicea, N., et al., The miRNA journey from theory to practice as a CNS biomarker. Front Genet, 2016. 7: p. 11.
6. Guan, Y., et al., MiRNA-196 is upregulated in glioblastoma but not in anaplastic astrocytoma and has prognostic significance. Clin Cancer Res, 2010. 16(16): p. 4289-97.
7. Gallego, J. A., et al., In vivo microRNA detection and quantitation in cerebrospinal fluid. J Mol Neurosci, 2012. 47(2): p. 243-8.
8. Cheng, L., et al., The detection of microRNA associated with Alzheimer's disease in biological fluids using next-generation sequencing technologies. Front Genet, 2013. 4: p. 150.
9. Freischmidt, A., et al., Systemic dysregulation of TDP-43 binding microRNAs in amyotrophic lateral sclerosis. Acta Neuropathol Commun, 2013. 1: p. 42.
10. Siegel, S. R., et al., Circulating microRNAs involved in multiple sclerosis. Mol Biol Rep, 2012. 39(5): p. 6219-25.
11. Ksiazek-Winiarek, D. J., M. J. Kacperska, and A. Glabinski, MicroRNAs as novel regulators of neuroinflammation. Mediators Inflamm, 2013. 2013: p. 172351.
12. Sheinerman, K. S. and S. R. Umansky, Circulating cell-free microRNA as biomarkers for screening, diagnosis and monitoring of neurodegenerative diseases and other neurologic pathologies. Front Cell Neurosci, 2013. 7: p. 150.
13. Cloutier, F., et al., MicroRNAs as potential circulating biomarkers for amyotrophic lateral sclerosis. J Mol Neurosci, 2015. 56(1): p. 102-12.
14. Galimberti, D., et al., Circulating miRNAs as potential biomarkers in Alzheimer's disease. J Alzheimers Dis, 2014. 42(4): p. 1261-7.
15. Hansen, K. F., K. Sakamoto, and K. Obrietan, MicroRNAs: a potential interface between the circadian clock and human health. Genome Med, 2011. 3(2): p. 10.
16. Weber, J. A., et al., The microRNA spectrum in 12 body fluids. Clin Chem, 2010. 56(11):p. 1733-41.
17. de Kloet, E. R., et al., Glucocorticoid signaling and stress-related limbic susceptibility pathway: about receptors, transcription machinery and microRNA. Brain Res, 2009. 1293: p. 129-41.
18. Wang, Y., et al., New players in the neuroendocrine system: A journey through the non-coding RNA world, in Molecular Neuroendocrinology: From Genome to Physiology. 2015, John Wiley & Sons, Ltd. p. 75-96.
19. Cheng, H.-Y. M., et al., microRNA Modulation of circadian-clock period and entrainment. Neuron, 2007. 54(5): p. 813-829.
20. Masotti, A., Interplays between gut microbiota and gene expression regulation by miRNAs. Frontiers in Cellular and Infection Microbiology, 2012. 2: p. 137.
21. Dalmasso, G., et al., Microbiota modulate host gene expression via microRNAs. PLoS One, 2011. 6(4): p. e19293.
22. O'Mahony, S. M., et al., Serotonin, tryptophan metabolism and the brain-gut-microbiome axis. Behav Brain Res, 2015. 277: p. 32-48.
23. Dickson, I., Gut microbiota: Intestinal microbiota oscillations regulate host circadian physiology. Nature Reviews Gastroenterology & Hepatology, 2017. 14(67).
24. Heegaard, N. H., et al., Diurnal variations of human circulating cell-free micro-RNA. PLoS One, 2016. 11(8): p. e0160577.
25. Hicks, S. D., et al., Salivary miRNA profiles identify children with autism spectrum disorder, correlate with adaptive behavior, and implicate ASD candidate genes involved in neurodevelopment. BMC Pediatr, 2016. 16: p. 52.
26. Hicks, S. D., et al., Overlapping microRNA expression in saliva and cerebrospinal fluid accurately identifies pediatric traumatic brain injury. J Neurotrauma, 2017.
27. Ainsworth, D., et al., k-SLAM: accurate and ultra-fast taxonomic classification and gene identification for large metagenomic data sets. Nucleic Acids Res, 2017. 45(4): p. 1649-1656.
28. Dhariwal, A., et al., MicrobiomeAnalyst: a web-based tool for comprehensive statistical, visual and meta-analysis of microbiome data. Nucleic Acids Res, 2017.
29. Ramsey, K. M., et al., Circadian clock feedback cycle through NAMPT-mediated NAD+ biosynthesis. Science, 2009. 324(5927): p. 651-4.
30. Bendova, Z., M. Sladek, and I. Svobodova, The expression of NR2B subunit of NMDA receptor in the suprachiasmatic nucleus of Wistar rats and its role in glutamate-induced CREB and ERK1/2 phosphorylation. Neurochem Int, 2012. 61(1): p. 43-7.
31. Wang, W. X., et al., Patterns of microRNA expression in normal and early Alzheimer's disease human temporal cortex: white matter versus gray matter. Acta Neuropathol, 2011. 121(2): p. 193-205.
32. Song, J. and Y. K. Kim, Identification of the role of miR-142-5p in Alzheimer's disease by comparative bioinformatics and cellular analysis. Front Mol Neurosci, 2017. 10: p.227.
33. Gorostiza, E. A., et al., Circadian pacemaker neurons change synaptic contacts across the day. Current Biology, 2014. 24(18): p. 2161-2167.
34. Jasinska, M., et al., Circadian rhythmicity of synapses in mouse somatosensory cortex. European Journal of Neuroscience, 2015. 42(8): p. 2585-2594.
35. Jasinska, M. and E. Pyza, Circadian plasticity of mammalian inhibitory interneurons. Neural Plast, 2017. 2017: p. 6373412.
36. Damdimopoulou, P., et al., A single dose of enterolactone activates estrogen signaling and regulates expression of circadian clock genes in mice. J Nutr, 2011. 141(9): p. 1583-9.
37. Laudes, M., et al., Visfatin/PBEF/Nampt and resistin expressions in circulating blood monocytes are differentially related to obesity and type 2 diabetes in humans. Horm Metab Res, 2010. 42(4): p. 268-73.
38. Figueroa, K. P., et al., Genetic variance in the spinocerebellar ataxia type 2 (ATXN2) gene in children with severe early onset obesity. PLoS One, 2009. 4(12): p. e8280.
39. Hanson, J. R., Terpenoids and steroids Vol. 7. 2007: Royal Society of Chemistry.
40. Son, G. H., et al., Adrenal peripheral clock controls the autonomous circadian rhythm of glucocorticoid by causing rhythmic steroid production. Proc Natl Acad Sci USA, 2008. 105(52): p. 20970-5.
41. Witwer, K. W., Circulating microRNA biomarker studies: pitfalls and potential solutions. Clin Chem, 2015. 61(1): p. 56-63.
42. Hicks, S. D. and F. A. Middleton, A comparative review of microRNA expression patterns in autism spectrum disorder. Front Psychiatry, 2016. 7: p. 176.
43. Liu, S., et al., The host shapes the gut microbiota via fecal microRNA. Cell Host Microbe, 2016. 19(1): p. 32-43.
44. Klingelhoefer, L. and H. Reichmann, Pathogenesis of Parkinson disease—the gut-brain axis and environmental factors. Nat Rev Neurol, 2015. 11(11): p. 625-36.
45. Li, Q. and J. M. Zhou, The microbiota—gut—brain axis and its potential therapeutic role in autism spectrum disorder. Neuroscience, 2016. 324(Supplement C): p. 131-139.
46. Carskadon, M. A., et al., Adolescent sleep patterns, circadian timing, and sleepiness at a transition to early school days. Sleep, 1998. 21(8): p. 871-81.
47. Wulff, K., et al., Sleep and circadian rhythm disruption in psychiatric and neurodegenerative disease. Nat Rev Neurosci, 2010. 11(8): p. 589-99.
48. Chen R, D'Alessandro M, Lee C. miRNAs are required for generating a time delay critical for the circadian oscillator. Curr Biol. 2013 Oct. 21; 23(20):1959-68.
49. Morgan X C, Huttenhower C(2012) Chapter 12:Human Microbiome analysis, PloSComputational Biology8(12):e1002808. doi: 10.1371/journal.pcbi.10028084.
50. Wood D E, Salzberg S L. Kraken: ultrafast metagenomic sequence classification using exact alignments. Genome Biol. 2014 Mar. 3; 15(3):R46.
51. Figueredo Dde S, Barbosa M R, Gitai DL, de Andrade T G. Predicted microRNAs for mammalian circadian rhythms. J Biol Rhythms. 2013 April; 28(2):107-16.
52. Mehta N, Cheng H Y. Micro-managing the circadian clock: The role of microRNAsin biological timekeeping. J Mol Biol. 2013 Oct. 9; 425(19):3609-24.

All publications and patent applications mentioned in this specification are herein incorporated by reference in their entirety to the same extent as if each individual publication or patent application was specifically and individually indicated to be incorporated by reference, especially referenced is disclosure appearing in the same sentence, paragraph, page or section of the specification in which the incorporation by reference appears. The citation of references herein does not constitute an admission that those references are prior art or have any relevance to the patentability of the technology disclosed herein. Any discussion of the content of references cited is intended merely to provide a general summary of assertions made by the authors of the references, and does not constitute an admission as to the accuracy of the content of such references.

1. (canceled)

2. A method for detecting or diagnosing a condition, disorder or disease associated with an abnormal diurnal or circadian rhythm in a human subject, the method comprising:
(a) determining a concentration level(s) of one or more micro RNAs (“miRNAs”) in a saliva sample taken from a human subject, and
(b) comparing the determined concentration level(s) of the one or more miRNAs against normal level(s) of the same one or more miRNAs in control human subject(s) not suffering from the condition, disorder of disease associated with abnormal diurnal or circadian rhythm,
(c) selecting a subject having an abnormal level of said one or more miRNAs as having or as being at higher risk for having a condition, disorder or disease associated with an abnormal diurnal or circadian rhythm;
wherein the one or more miRNAs is selected from the group consisting of miR-24-3p, miR-200b-3p, miR-203a-3p, miR-26a-5p, hsa-miR-106b-3p, hsa-miR-128-3p, hsa-miR-130a-3p, hsa-miR-15a-5p, hsa-miR-192-5p, hsa-miR-199a-3p, hsa-miR-199b-3p, hsa-miR-221-3p, hsa-miR-26b-5p, hsa-miR-3074-5p, hsa-miR-30e-3p, hsa-miR-320a, hsa-miR-345-5p, hsa-miR-375, hsa-miR-423-3p, hsa-miR-92a-3p, hsa-miR-93-5p, hsa-let-7a-5p, hsa-let-7d-3p, hsa-miR-101-3p, hsa-miR-10b-5p, hsa-miR-125b-2-3p, hsa-miR-1307-5p, hsa-miR-140-3p, hsa-miR-142-3p, hsa-miR-143-3p, hsa-miR-148b-3p, hsamiR-16-5p, hsa-miR-181a-5p, hsa-miR-181c-5p, hsa-miR-186-5p, hsa-miR-191-5p, hsa-miR-193a-5p, hsa-miR-205-5p, hsa-miR-215-5p, hsa-miR-21-5p, hsa-miR-223-3p, has-miR-22-3p, hsa-miR-23a-3p, hsa-miR-23b-3p, hsa-miR-25-3p, hsa-miR-29a-3p, hsa-miR-30d-5p, hsa-miR-320b, hsa-miR-361-5p, hsa-miR-363-3p, hsa-miR-374a-3p, hsa-miR-423-5p, hsa-miR-425-5p, hsa-miR-532-5p, hsa-miR-574-3p, hsa-miR-629-5p, hsa-miR-98-5p and/or those miRNA which share the seed sequences thereof.

3. The method of claim 2, wherein values of said miRNA concentration level(s) are normalized to an expression level, or average expression level, of one or more housekeeping genes whose RNA expression level is substantially invariant; and/or wherein said miRNA concentration levels are normalized to compensate for diurnal or circadian fluctuations in the expression of the one or more miRNA levels, normalized to compensate for fluctuations in the expression of the one or more miRNA levels due to food intake or exercise that raises the heart rate; or adjusted to compensate for differences in age, sex or genetic background.

4. The method of claim 2, wherein (a) determining a concentration of one or more miRNAs is done by RNA sequencing (“RNA-seq”), qPCR, a miRNA array, or multiplex miRNA profiling.

5. The method for detecting or diagnosing of claim 2, wherein said one or more miRNAs are selected from the group consisting of miR-142-5p, miR-130b-3p, miR-629-5p, miR-140-3p, miR-128-3p, miR-181c-5p, miR345-5p, miR-22-5p, miR-8089, miR-221-3p, and miR-200b-5p.

6. The method of claim 2, wherein the saliva sample is taken from a human subject suspected of having a sleep disorder or disordered sleep and wherein the miRNAs are selected from the group consisting of at least one of miR-24-3p, miR-200b-3p, miR-203a-3p, and miR-26a-5p.

7. The method of claim 2, wherein the saliva sample is taken from the human subject at a particular time of day and the concentration level(s) of miRNA in said sample are compared to normal miRNA values in saliva taken at the same time of day under otherwise identical conditions.

8. The method of claim 2, wherein the saliva sample is taken from the human subject at a different time of day than the time of day at which the normal level(s) of miRNAs were determined, further comprising adjusting or normalizing the value of the miRNA level(s) determined in the saliva sample to compensate for diurnal or circadian fluctuations in miRNA level(s).

9. The method of claim 2, wherein the saliva sample is taken from the human subject at a different time of day than the time of day at which the normal level(s) of miRNAs were determined, further comprising adjusting or normalizing the value of the miRNA level(s) determined in the saliva sample to compensate for diurnal or circadian fluctuations in miRNA level(s) as determined by a regression model or other statistical analysis; or to compensate for age, sex, or genetic background.

10. The method of claim 2, wherein the saliva sample is taken within 1 hour of waking, before brushing or rinsing the mouth, before eating or drinking, and/or before exercise that elevates heart rate.

11. The method of claim 2, wherein said selecting comprises selecting a subject having abnormal levels of four or more of said miRNAs, and, optionally calculating a Pearson correlation coefficient of said abnormal miRNA levels with likelihood of an at least one symptom of a condition, disorder, or disease associated with an abnormal diurnal or circadian rhythm.

12. The method of claim 2, wherein said selecting comprises selecting a subject having abnormal levels of ten or more of said miRNAs, and, optionally calculating a Pearson correlation coefficient of said abnormal miRNA levels with likelihood of an at least one symptom of a condition, disorder, or disease associated with an abnormal diurnal or circadian rhythm.

13. The method of claim 2, further comprising determining an expression level of RNA(s) in said subject from one or more salivary microbes selected from the group consisting of Falconid herpesvirus, Prevotella melaninogenica ATCC 25845, Haemophilus parainfluenzae T3T1, Veillonella parvula DSM 2008, Macrococcus caseolyticus JSCC5402, Fusobaterium nucleatum subsp. nucleatum 25586, Haemophilus, Fusobacterium nucleatum subsp. vincentii, Mason-Pfizer monkey virus, Camplyobacer hominis ATCC, and Prevotella; or a microbe having a genome that is at least 90, 95, 96, 97, 98, 99, 99.5 or 100% similar or identical thereto; and comparing the expression level(s) of the microbial RNAs against normal level(s) of the same one or more microbial RNAs, wherein the normal (or control) expression level is that found in a subject, an average from two of more subjects, not having a condition, disorder, or disease associated with an abnormal diurnal or circadian rhythm; or concentration level(s) determined in the subject prior to appearance of one or more symptoms of a condition, disorder, or disease associated with an abnormal diurnal or circadian rhythm; and further selecting a subject having an abnormal expression level of said one or more microbial RNAs as having or as being at higher risk for having said condition, disorder or disease.

14. The method of claim 13, wherein determining salivary miRNA levels or determining microbial RNA expression level(s) is done by RNA Sequencing (“RNA-Seq”).

15. The method of claim 13, wherein the sequencing data raw read counts are quantile-normalized, mean-centered, and divided by the standard deviation of each variable; data are normalized to account for inter-sample count variations; and/or wherein data are normalized to expression of one or more invariant miRNAs to describe relative and/or absolute expression levels; and optionally further statistically analyzing the normalized data.

16. The method of claim 2, further comprising treating a subject having at least one abnormal level of miRNA or microbial RNA expression level characteristic of a condition, disorder, or disease associated with an abnormal diurnal or circadian rhythm with a regimen that reduces the at least one abnormal salivary level of one or more miRNAs and/or reduces one or more abnormal microbial RNA expression levels.

17. The method of claim 16, further comprising obtaining saliva samples on at least two different points in time and determining efficacy of a treatment regimen when said second or subsequent saliva sample has miRNA level(s) and/or microbial RNA expression levels closer to normal.

18. The method of claim 2, further comprising treating a subject with a regimen that reduces at least one abnormal salivary level of one or more miRNAs or one or more abnormal microbial RNA expression levels characteristic of a condition, disorder or disease associated with an abnormal diurnal or circadian rhythm in a human subject, wherein said regimen comprises administering one or more of a sleep disorder therapy, a drug therapy, a miRNA or miRNA antagonist therapy, antimicrobial therapy, diet or nutritional therapy, phototherapy, psychotherapy, a behavior therapy, a communication therapy or an alternative medical therapy, wherein the subject was identified as having symptoms of a condition, disorder or disease associated with an abnormal diurnal or circadian rhythm.

19. An miRNA assay kit for detecting miRNAs comprising one, two or more probes or primers complementary to or otherwise suitable for amplification and/or detection of miRNAs selected from the group consisting of miR-24-3p, miR-200b-3p, miR-203a-3p, miR-26a-5p, hsa-miR-106b-3p, hsa-miR-128-3p, hsa-miR-130a-3p, hsa-miR-15a-5p, hsa-miR-192-5p, hsa-miR-199a-3p, hsa-miR-199b-3p, hsa-miR-221-3p, hsa-miR-26b-5p, hsa-miR-3074-5p, hsa-miR-30e-3p, hsa-miR-320a, hsa-miR-345-5p, hsa-miR-375, hsa-miR-423-3p, hsa-miR-92a-3p, hsa-miR-93-5p, hsa-let-7a-5p, hsa-let-7d-3p, hsa-miR-101-3p, hsa-miR-10b-5p, hsa-miR-125b-2-3p, hsa-miR-1307-5p, hsa-miR-140-3p, hsa-miR-142-3p, hsa-miR-143-3p, hsa-miR-148b-3p, hsamiR-16-5p, hsa-miR-181a-5p, hsa-miR-181c-5p, hsa-miR-186-5p, hsa-miR-191-5p, hsa-miR-193a-5p, hsa-miR-205-5p, hsa-miR-215-5p, hsa-miR-21-5p, hsa-miR-223-3p, has-miR-22-3p, hsa-miR-23a-3p, hsa-miR-23b-3p, hsa-miR-25-3p, hsa-miR-29a-3p, hsa-miR-30d-5p, hsa-miR-320b, hsa-miR-361-5p, hsa-miR-363-3p, hsa-miR-374a-3p, hsa-miR-423-5p, hsa-miR-425-5p, hsa-miR-532-5p, hsa-miR-574-3p, hsa-miR-629-5p, and hsa-miR-98-5p; reagents for amplification and/or detection of said miRNAs, and optionally a reaction substrate or platform, packaging materials and/or instructions for use.

20. The assay kit of claim 19 for diagnosis or detection of a sleep disorder, wherein said assay kit detects at least one of miR-24-3p, miR-200b-3p, miR-203a-3p, or miR-26a-5p.

21. (canceled)

22. A method for identifying a miRNA, a concentration of which in human saliva, fluctuates according to a diurnal or circadian rhythm, comprising:
(a) collecting saliva samples from one or more subjects at 2, 3, 4, 5, 6, 7, 8, 9, 10, 11, 12 or more times or intervals during a 24 hour period,
(b) sequencing miRNA in said samples,
(c) identifying differently expressed miRNAs by counting sequencing reads per miRNA, normalizing sequence read data, and comparing normalized sequence read counts among saliva samples taken at different times,
(d) normalizing sequence read data to RNA expression of a housekeeping gene or miRNA (which exhibits invariant expression over a 24 hour period), or to an averaged RNA expression from two or more housekeeping genes,
(e) performing a multivariate regression analysis or other statistical analysis on the normalized RNA expression data from different time points or intervals,
(f) optionally, calculating a Pearson correlation coefficient for data obtained describing concentration levels of one or more miRNAs and one or more RNA expression levels from a microorganism found in saliva,
(g) selecting one or more miRNAs as having an expression level that fluctuates according to a diurnal or circadian rhythm; and
(h) optionally, determining target genes for miRNAs using DIANA miRpath or other software.
